(19) United States
(12) Patent Application Publication
(10) Pub. No.: US 2025/0375455 A1
(43) Pub. Date: Dec. 11, 2025

(54) TREATMENT OF TUBERCULOSIS

(71) Applicants: CHARIOT INNOVATIONS LTD, London (GB); EMORY UNIVERSITY, Atlanta, GA (US); THE UNIVERSITY OF LIVERPOOL, Merseyside (GB)

(72) Inventors: Leopold TIENTCHEU, London (GB); Schadrac AGBLA, Liverpool (GB); Daniel KALMAN, Atlanta, GA (US)

(73) Assignees: CHARIOT INNOVATIONS LTD, London (GB); EMORY UNIVERSITY, Atlanta, GA (US); THE UNIVERSITY OF LIVERPOOL, Liverpool (GB)

(21) Appl. No.: 18/874,803

(22) PCT Filed: Jun. 13, 2023
(86) PCT No.: PCT/GB2023/051542

§ 371 Date: Dec. 13, 2024

(30) Foreign Application Priority Data
Jun. 13, 2022 (GB) 2208654.0

(51) Int. Cl. A61K 31/5377; A61K 31/506; A61K 31/517; A61P 31/06

(52) U.S. Cl. CPC A61K 31/5377; A61K 31/506; A61K 31/517; A61P 31/06

(57) ABSTRACT

The present invention is directed to a protein kinase inhibitor for use in the treatment of tuberculosis, by direct inhibition of the growth of a Mycobacterium tuberculosis complex species, and to corresponding methods of treating tuberculosis. More specifically, the invention relates to the use of protein tyrosine kinase inhibitors such as gefitinib, erlotinib, or imatinib for use in the treatment of tuberculosis, by direct inhibition of the growth of a Mycobacterium tuberculosis complex species, rather than through use in host-directed therapy. Also provided are methods of determining the ability of a protein tyrosine kinase inhibitor to inhibit the growth of a Mycobacterium tuberculosis complex species and methods of identifying a protein tyrosine kinase inhibitor as being potentially effective in the treatment of tuberculosis by direct inhibition of a Mycobacterium tuberculosis complex species.

### FIELD OF THE INVENTION

The present invention relates to protein tyrosine kinase inhibitors for use in the treatment of tuberculosis. In particular the protein tyrosine kinase inhibitors are used to directly inhibit growth of Mycobacterium tuberculosis complex species, and thereby treat tuberculosis.

### Introduction

Tuberculosis, caused by members of the Mycobacterium tuberculosis complex (MTBC), claimed 1.4 million lives and caused 10 million new cases worldwide in 2020 (WHO report 2021). TB remains a severe public health threat globally despite the wide use of antibiotic drugs developed over five decades ago. The treatment of drug-susceptible TB takes six months. It uses four antibiotics, including two months with isoniazid (INH), rifampicin (RMP), ethambutol (EMB) and pyrazinamide (PZA), followed by four months with INH and RMP. This lengthy treatment promotes the development of multidrug-resistant TB (MDR TB) associated with poor compliance. MDR TB treatment is more complex, lasting up to 24 months, and uses toxic drugs, including injectables. Thus, shortening TB treatment can improve compliance and significantly reduce the development of MDR TB.

This goal requires innovative therapeutics such as adjunctive treatment with drugs targeting patients' immunity, so-called host-directed therapeutics (or HDTs). HDTs can augment anti-TB immunity, reduce immunopathology, enhance the therapeutic effect of anti-bacillary medicines, and reduce the drug pressure favouring resistance to develop (Korbee, Heemskerk et al. 2018). Since HDTs mainly target the host rather than the bacteria, they can shorten the length of both drug-susceptible and MDR TB treatment Fields (Chandra, Rajmani et al. 2016, Gehre, Otu et al. 2016, Korbee, Heemskerk et al. 2018).

Despite the promise of these clinical developments, there remains a need for new and effective methods of treating tuberculosis.

### SUMMARY OF THE INVENTION

In a first aspect, the invention provides a protein tyrosine kinase inhibitor for use in the treatment of tuberculosis, by direct inhibition of the growth of a Mycobacterium tuberculosis complex species.

In a second aspect, the invention provides gefitinib for use in the treatment of tuberculosis, by direct inhibition of growth of a Mycobacterium tuberculosis complex species. The gefitinib may be for use as a broad-spectrum tuberculosis treatment.

In a third aspect, the invention provides erlotinib for use in the treatment of tuberculosis, by direct inhibition of growth of a Mycobacterium tuberculosis complex species.

In a fourth aspect, the invention provides imatinib for use in the treatment of tuberculosis, by direct inhibition of growth of a Mycobacterium tuberculosis complex species.

In a fifth aspect, the invention provides gefitinib for use in the treatment of tuberculosis, wherein the tuberculosis has been identified as caused by infection with Mycobacterium tuberculosis-lineage2.

In a sixth aspect, the invention provides erlotinib for use in the treatment of tuberculosis, wherein the tuberculosis has been identified as caused by infection with a Mycobacterium tuberculosis complex species selected from the group consisting of: Mycobacterium bovis; Mycobacterium tuberculosis; and Mycobacterium africanum.

In a seventh aspect, the invention provides imatinib for use in the treatment of tuberculosis, wherein the tuberculosis has been identified as caused by infection with a Mycobacterium tuberculosis complex species selected from the group consisting of: Mycobacterium bovis; Mycobacterium africanum and Mycobacterium tuberculosis.

In an eighth aspect, the invention provides a method of treating tuberculosis, the method comprising providing to a subject requiring such treatment an amount of a protein tyrosine kinase inhibitor that is sufficient to directly inhibit of growth of a Mycobacterium tuberculosis complex species.

In a ninth aspect, the invention provides a method of determining the ability of a protein tyrosine kinase inhibitor to inhibit the growth of a Mycobacterium tuberculosis complex species, the method comprising:
incubating a sample of the protein tyrosine kinase inhibitor with a clinically relevant Mycobacterium tuberculosis complex species; and
assessing growth of the Mycobacterium tuberculosis complex species by means of an assay selected from the group consisting of: an assay that directly measures bacterial growth; an assay that measures bacterial growth via accumulation of a reporter; and an assay that measures actively multiplying bacteria; wherein
   a reduction of the growth of the Mycobacterium tuberculosis complex species as assessed by one or more of the assays indicates that the protein tyrosine kinase inhibitor is able to inhibit growth of the Mycobacterium tuberculosis complex species.

In a tenth aspect, the invention provides a method of identifying a protein tyrosine kinase inhibitor as being potentially effective in the treatment of tuberculosis by direct inhibition of a Mycobacterium tuberculosis complex species, the method comprising:
incubating a sample of the protein tyrosine kinase inhibitor with a clinically relevant Mycobacterium tuberculosis complex species; and
assessing growth of the Mycobacterium tuberculosis complex species by means of an assay selected from the group consisting of: an assay that directly measures bacterial growth; an assay that measures bacterial growth via accumulation of a reporter; and an assay that measures actively multiplying bacteria; wherein
   inhibition of the growth of the Mycobacterium tuberculosis complex species as assessed by one or more of the assays indicates that the protein tyrosine kinase inhibitor is potentially effective in the treatment of tuberculosis by direct inhibition of the Mycobacterium tuberculosis complex species.

### FIGURE LEGENDS

FIG. 1 shows a plate plan used in conducting the studies set out in the Examples.

FIG. 2 illustrates growth rate differences between reporter-gene-tagged M. tuberculosis complex lineages. Clinical isolates from West African TB patients transfected with fluorescent and luminescent plasmid representing M. tuberculosis lineage2 (circle), M. tuberculosis lineage4 (square), M. bovis (triangle), M. africanum lineage5 (diamond) and M. africanum lineage6 (hexagon) were cultured into 7H9 Middlebrook media with daily growth measurement over 20 days. (A) Ongoing cultures of Mtb-lineage2 (circle) and Mtb-lineage4 (square) were plated every two days, and the colony-forming units were read weekly until they became confluent. Each dots represent the predicted mean and 95% confidence interval (95% CI) of three individual experiments. (B) Represent the bacilli culture daily light absorbance at OD600 nm converted into Transmittance. (C) Represent the fluorescence of accumulated reporter plasmid GFP, and (D) is the daily constitutively expressed luminescence of actively multiplying MTBC lineages. Dots and bars represent the daily predicted mean, 95% CI of 6 replicate wells per plate and three consecutive experiments.

FIG. 3 demonstrates that host-directed therapeutic drugs direct inhibition of M. tuberculosis complex lineages testing based on colony-forming units. Mtb-lineage2 (a) and Mtb-lineage4 (b) were without drug circle=bacteria+drug diluent DMSO (control) or in the presence of different concentrations (square=5 μM; triangle=10 μM, star=50 μM) of Erlotinib, Gefitinib and Imatinib respectively. Aliquots were taken for serial dilution and plating in a 7H11 agar plate every two days for 16 days to evaluate HDTs drug direct inhibition compared to the control without drug. Significant p-value were captured as *=p<0.05-0.01.

FIG. 4 illustrates Absorbance (Transmittance) readout of host-directed therapeutic drugs direct inhibition of M. tuberculosis complex lineages. [I] Example of daily measurement of the absorbance at OD600 nm (transformed into transmittance [Absorbance=−Log₁₀ Transmittance]) of M. tuberculosis lineage4 (a), M. africanum lineage5 (b) and M. tuberculosis lineage2 (c) in presence of different concentrations (square=5 μM; triangle=10 μM, star=Green=50 μM) of Imatinib and Gefitinib respectively over 20 days. Controls conditions include circle=bacteria+drug diluent DMSO; diamond=bacteria+Isoniazid 0.4 μg/mL and hexagon=bacteria+Rifampicin 1 μg/mL. [II] Summary plots of percentage bacteria inhibition by different concentrations (5 μM, 10 μM and 50 μM) of Erlotinib (circle), Gefitinib (square) and Imatinib (triangle). Each plot represents one of the five lineages tested, including a) M. tuberculosis lineage4, b) M. africanum lineage6, c) M. tuberculosis Lineage2, d) M. africanum lineage5, and e) M. bovis. P-value represents the growth difference between the bacteria without the drug (control) and in the presence of different drug concentrations over 20 days. *=p<0.05-0.01, **=p<0.01-0.001, ***=p<0.001. The p-values are ordered according to the tested HDT drug curve.

FIG. 5 illustrates fluorescence readout of host-directed therapeutic drugs direct inhibition of M. tuberculosis complex lineages. [I] Example of daily measurement of accumulated GFP fluorescence of M. tuberculosis lineage4 (a), M. africanum lineage6 (b) and M. tuberculosis lineage2 (c) in presence of different concentrations (square=5 μM; triangle=10 μM, star=Green=50 μM) of Imatinib and Gefitinib respectively. Controls conditions include circle=bacteria+drug diluent DMSO; diamond=bacteria+Isoniazid 0.4 μg/mL; hexagon=bacteria+Rifampicin 1 μg/mL; crossed-circle=7H9 culture media only and crossed-square=No bacteria+HDT 50 uM. P-value represent the growth difference between the growth of the bacteria without drug and in the presence of different drug concentration over 20 days. *=p<0.05-0.01, **=p<0.01-0.001, ***=p<0.001-0.0001, ****=p<0.0001. [II] Summary plots of percentage bacteria inhibition by different concentrations (5 μM, 10 μM and 50 μM) of Erlotinib (circle), Gefitinib (square) and Imatinib (triangle). Each plot represents one of the five lineages tested, including a) M. tuberculosis lineage4, b) M. africanum lineage6, c) M. tuberculosis Lineage2, d) M. africanum lineage5, and e) M. bovis. P-value represents the growth difference between the growth of the bacteria without the drug and in the presence of different drug concentrations over 20 days. *=p<0.05-0.01, **=p<0.01-0.001, ***=p<0.001. The p-values are ordered according to the tested HDT drug curve.

FIG. 6 illustrates luminescence readout of host-directed therapeutic drugs direct inhibition of M. tuberculosis complex lineages. [I] Example of daily measurement of constitutively expressed luminescence of M. tuberculosis lineage4 (a), M. africanum lineage6 (b) and M. tuberculosis lineage2 (c) in presence of different concentrations (square=5 μM; triangle=10 μM, star=Green=50 μM) of Imatinib and Gefitinib respectively. Controls conditions include circle=bacteria+drug diluent DMSO; diamond=bacteria+Isoniazid 0.4 μg/mL; hexagon=bacteria+Rifampicin 1 μg/mL; crossed-circle=7H9 culture media only and crossed-square=No bacteria+HDT 50 uM. P-value represent the growth difference between the growth of the bacteria without drug and in the presence of different drug concentration over 20 days. *=p<0.05-0.01, **=p<0.01-0.001, ***=p<0.001-0.0001, ****=p<0.0001. [II] Summary plots of percentage bacteria inhibition by different concentrations (5 μM, 10 μM and 50 μM) of Erlotinib (circle), Gefitinib (square) and Imatinib (triangle). Each plot represents one of the five lineages tested, including a) M. tuberculosis lineage4, b) M. africanum lineage6, c) M. tuberculosis Lineage2, d) M. africanum lineage5, and e) M. bovis. P-value represents the growth difference between the growth of the bacteria without the drug and in the presence of different drug concentrations over 20 days. *=p<0.05-0.01, **=p<0.01-0.001, ***=p<0.001. The p-values are ordered according to the tested HDT drug curve.

FIG. 7 sets out normalised percentage growth rate of bacteria in different TKI concentrations. Different Imatinib, Erlotinib, and Gefitinib concentrations were added to the M. tuberculosis lineage2 cultures. The Y-axis shows the bacteria growth rate at day 12 and the X-axis the Log₁₀ of the TKI concentrations. The Gompertz equation was used to derive the MIC, and a four-parameter logistic equation was used to derive the IC50 in Graph Pad Prism software.

FIG. 8 illustrates normalised percentage growth rate of bacteria in different Imatinib concentrations. Different Imatinib concentrations were added to all MTBC lineages tested. The Y-axis shows the bacteria growth rate at day 12 and the X-axis the Log₁₀ of the TKI concentrations. The Gompertz equation was used to derive the MIC, and a four-parameter logistic equation was used to derive the IC50 in Graph Pad Prism software.

### DETAILED DESCRIPTION OF THE INVENTION

The present invention is based upon the inventors' surprising finding that protein tyrosine kinase inhibitors, a class of compounds that have previously been suggested to be useful in the treatment of tuberculosis by host-directed therapy (HDT), also possess the ability to directly inhibit growth of species of the Mycobacterium tuberculosis complex (MTBC).

The results presented here illustrate that the ability to directly inhibit growth of MTBC species is present in a number of different classes of protein tyrosine kinase inhibitors. This property has been demonstrated in respect of inhibitors of a number of protein tyrosine kinases. These include inhibitors of the protein tyrosine kinase associated with the EGFR, and inhibitors of the protein tyrosine kinase associated with the PDGFR. The inventors have demonstrated that the protein tyrosine kinase inhibitors gefitinib, erlotinib, and imatinib all possess the ability to directly inhibit growth of members of the MTBC.

As the skilled person will appreciate, this direct inhibition of MTBC growth can be used to treat tuberculosis directly, rather than via a host directed response. The inventors' finding is unexpected, and opens the possibility of using these compounds in new ways to achieve clinically useful outcomes, as discussed further below.

By identifying protein tyrosine kinase inhibitors' ability to directly inhibit growth of MTBC species, the inventors had recognised that these compounds can be used in a number of situations where they would not previously have been considered suitable.

As discussed further below, the inventors believe that their findings suggest a previously unrecognised use for protein tyrosine kinase inhibitors in the treatment of tuberculosis in immunocompromised subjects.

Treatments aimed at harnessing the direct growth inhibitory effect of protein tyrosine kinase inhibitors on MTBC species may also be expected to achieve faster results than prior art uses as HDTs, where clinical effect is reliant upon a response by the recipient's own cells.

The newly identified role of protein tyrosine kinase inhibitors as direct inhibitors of MTBC species' growth also suggests other new ways in which these agents can be used therapeutically, for example in combination treatments, or in the treatment of tuberculosis associated with infection by multidrug resistant forms of MTBCs.

Perhaps most importantly, the inventors have found that different inhibitors have greater or lesser impact upon different MTBC species or lineages. This allows, for the first time, the development of patient stratification approaches in which the agent used for treatment can be tailored to meet a patient's needs based on the MTBC species or lineage with which they have been infected. Therapeutic agents shown to offer benefits in treatment can be selected, and unnecessary treatment with agents likely to prove unsuccessful avoided.

It will be appreciated that an approach in which patient stratification is based upon identification of the MTBC species or lineage with which a patient is infected would not be considered relevant in previous therapeutic approaches (using kinase inhibitors as HDTs), as in these the response of the patient's own cells is considered the primary factor determining therapeutic effectiveness.

The ways in which the protein tyrosine kinase inhibitors may be used based upon this information will also differ from those previously described. For example, the inventors' results suggest that the direct MTBC growth inhibitory activity of protein tyrosine kinase inhibitors may be effective, and indeed even most effective, at doses that are lower than those previously proposed for HDT uses. The ability to achieve therapeutic effects using lower doses of protein tyrosine kinase inhibitors offers many advantages including in terms of the costs of treatment and the reduced risk of side effects or off-target activities.

To at least some extent, this identification of the directly growth inhibitory activities of protein tyrosine kinase inhibitors arises from the inventors' determining that commonly used assays for MTBC growth, and in particular colony-forming unit (CFU)-based assays are not sufficiently sensitive to adequately report on relevant changes in bacterial growth rates. This gives rise to the ninth and tenth aspects of the invention.

The invention will now be further defined with reference to the definitions and examples set out below.

### Tuberculosis and Treatment of Tuberculosis

Tuberculosis (TB) is an infectious disease of the lungs caused by bacteria such as Mycobacterium tuberculosis, and other species in the Mycobacterium tuberculosis complex.

The infection may exist in latent or active forms. Estimates suggest that up to a quarter of the world's population may have latent TB. Approximately 10% of latent TB infections progress to active TB, and around half of active TB infections prove deadly if untreated.

For the purposes of the present invention, “treatment of tuberculosis” may be considered as any partial or complete alleviation of a subject's symptoms associated with active tuberculosis, or of the underlying bacterial infection giving rise to latent or active tuberculosis.

The medical uses and methods of treatment defined herein are particularly useful in the treatment of active tuberculosis.

### Species of the Mycobacterium Tuberculosis Complex, and Their Lineages

The Mycobacterium tuberculosis complex, also referred to as the MTBC, comprises a number of genetically related species of Mycobacterium, all of which are capable of causing tuberculosis. Particular members of the MTBC may be specifically associated with tuberculosis in specific locations, or in specific hosts (whether humans, or other animals). The MTBC includes the following Mycobacterium species: Mycobacterium tuberculosis; Mycobacterium africanum; Mycobacterium bovis; Mycobacterium orygis; Mycobacterium microti; Mycobacterium canetti; Mycobacterium caprae; Mycobacterium pinnipedii; Mycobacterium suricattae; and Mycobacterium mungi.

Mycobacterium tuberculosis and Mycobacterium africanum primarily cause tuberculosis in humans, while the other members of the MTBC were identified as causing tuberculosis in other species, but may also infect humans. For example, Mycobacterium bovis is responsible for bovine tuberculosis in cattle, but can also cause human infections.

A number of clinically relevant lineages have also been identified within the species of the MTBC, particularly Mycobacterium tuberculosis and Mycobacterium africanum (which may respectively be abbreviated to Mtb and Maf in this context).

Mycobacterium tuberculosis-lineage 1 is associated with tuberculosis in The Philippines and rim of the Indian Ocean. Mycobacterium tuberculosis-lineage2 is associated with disease in Beijing or East Asia. Mycobacterium tuberculosis-lineage 3 is associated with tuberculosis in India and East Africa, while Mycobacterium tuberculosis-lineage4 is associated with the disease in Europe, America and Africa. Mycobacterium africanum-lineage5 is also referred to as West African Type1, while Mycobacterium africanum-lineage6 is also referred to as West African Type2. More recently the list has been expanded to include Mycobacterium tuberculosis-lineage7 associated with tuberculosis in Ethiopia, Mycobacterium tuberculosis lineage8 and Mycobacterium africanum-lineage9 that are both rare lineages found in Africa.

Except where context requires otherwise, references to a species of the MTBC may be taken as relating to any of the species or lineages referred to above. However, the inventors have noticed that particular species and lineages of the MTBC differ in their responses to different protein tyrosine kinase inhibitors. These differential responses, which could not have been predicted prior to the inventors' studies, give rise to certain aspects of the invention described herein.

As described elsewhere in the present disclosure, the medical uses and methods of the invention may be of particular use in the treatment of tuberculosis caused by species of the MTBC selected from the group consisting of: Mycobacterium tuberculosis, Mycobacterium africanum, and Mycobacterium bovis. Within these species, the uses and methods of the invention may be particularly applicable to Mycobacterium tuberculosis-lineage2 and Mycobacterium tuberculosis-lineage4, and to Mycobacterium africanum-lineage5; and Mycobacterium africanum-lineage6. Within these lineages, the uses and methods of the invention have been proven to be effective in respect of each of the sublineages tested: Mycobacterium tuberculosis-lineage2 sublineage East Asia and Mycobacterium tuberculosis-lineage4 sublineage Cameroon, Haarlem, LAM or Euro-American and to Mycobacterium africanum-lineage5 sublineage West Africa 1; and Mycobacterium africanum-lineage6 sublineage West Africa 2. Thus, the results demonstrate that the sublineages tested within these particular lineages have proved to be consistent in terms of their response to the protein tyrosine kinase inhibitors.

In the case that tuberculosis is caused by an infection with Mycobacterium tuberculosis (such as with Mycobacterium tuberculosis-lineage2 or Mycobacterium tuberculosis-lineage4), the inventors' results indicate that a suitable protein tyrosine kinase inhibitor may be selected from the group consisting of: gefitinib; erlotinib; and imatinib.

In contrast, when tuberculosis is caused by an infection with Mycobacterium africanum (such as with Mycobacterium africanum-lineage5 or Mycobacterium africanum-lineage6), a suitable protein tyrosine kinase inhibitor may be selected from the group consisting of: gefitinib; imatinib and erlotinib.

When tuberculosis is caused by an infection with Mycobacterium bovis, the results indicate that a suitable protein tyrosine kinase inhibitor may be selected from the group consisting of: gefitinib; erlotinib; and imatinib.

### Direct Inhibition of Growth of MTBC Species

The ability of protein tyrosine kinase inhibitors to directly inhibit growth of MTBC species represents a newly identified biological effect arising from the activity of these agents. This indicates a new mode of action by which protein tyrosine kinase inhibitors, such as gefitinib, erlotinib and imatinib, can bring about treatment of tuberculosis.

Previous reports on the effect of protein tyrosine kinase inhibitors on tuberculosis have focused on the ability of these agents to act as HDTs. Only now, through the inventors' work disclosed for the first time in this application, has it been found that these compounds can also exert a direct therapeutic influence on the MTBC species responsible for tuberculosis. This is demonstrated in the Examples, where, even in the absence of host cells, protein tyrosine kinase inhibitors are able to inhibit the growth of MTBC species in culture. This indicates that the compounds are having a direct inhibitory effect on growth of the MTBC species, without their effect needing to be mediated by a host (for example via an upregulated or modified immune response). As referred to elsewhere in this specification, the inventors believe that a lack of sensitivity in the colony-forming unit (CFU) assay most commonly used in this field has contributed to the fact that this property has not been noted before.

As discussed in more detail elsewhere in this disclosure, the doses of protein tyrosine kinase inhibitors that may be employed in the medical uses and methods of treatment of the present invention, making use of direct inhibition of growth of MTBC species, are readily distinguished from the doses that have been used or suggested in the context of HDT use. In particular, the present invention allows the use of doses of protein tyrosine kinase inhibitors that are lower than has previously been thought necessary in order to achieve a therapeutic effect.

Furthermore, since the direct inhibition of the growth of MTBC species causing tuberculosis that underpins the medical uses and methods of the invention does not rely upon a subject's own immune response to kill mycobacteria, the medical uses and methods of the invention can be of clinical benefit to subjects that would not be expected to gain from HDT treatment.

### Treatment of Immunocompromised Subjects

Merely by way of example, the inventors finding that protein tyrosine kinase inhibitors such as gefitinib, erlotinib and imatinib may be used to directly inhibit growth of MTBC species means that the skilled person will now recognise that such agents may be used to treat tuberculosis in an immunocompromised subject. Suitably, such a subject may be immunocompromised due to a medical condition, such as an HIV infection. HIV infection is commonly associated with active tuberculosis.

Alternatively, or additionally, the subject may be immunocompromised due to undergoing treatment with immunosuppressive drugs.

### Combination Treatments

Identification of the direct growth inhibitory effects of protein tyrosine kinase inhibitors on MTBC species also suggests ways in which this property may be combined with other therapeutic modalities in order to achieve effective combination treatments for tuberculosis.

By way of example, this suggests that the medical uses and methods of the invention may make use of a protein tyrosine kinase inhibitor in combination with an antibiotic. Suitably, an antibiotic for use in such a combination treatment may be selected from the group consisting of: isoniazid; rifampicin; ethambutol; and pyrazinamide. Each of these antibiotics have been shown to be effective in the treatment of tuberculosis, for example in the well-established 2HREZ4HR treatment pattern, involving two months of treatment with each of these antibiotics, followed by four months of treatment with isoniazid and rifampicin. Augmenting the activities of these antibiotics with the direct inhibition of MTBC growth achieved by protein tyrosine kinase inhibitors can be expected to improve the effectiveness of the antibiotic treatment.

As noted above, current antibiotic treatment for tuberculosis may require administration of antibiotics for protracted periods. The further direct anti-tuberculosis activity demonstrated by protein tyrosine kinase inhibitors in the medical uses and methods of treatment of the invention may allow effective therapy to be achieved using reduced courses of treatment involving less time receiving antibiotics.

The advantages discussed above may be expected to be able to be achieved by use of any protein tyrosine kinase inhibitor (such as gefitinib, erlotinib or imatinib) with an antibiotic agent, but the broad-spectrum activity demonstrated by gefitinib in the inventors' studies make this agent especially suitable for such medical uses and methods of treatment.

### Protein Tyrosine Kinase Inhibitors

Protein kinases are enzymes that cause the phosphorylation of protein substrates. This phosphorylation modifies the activity or function of the protein. Protein tyrosine kinases specifically phosphorylate tyrosine amino acid residues within a protein substrate.

Protein tyrosine kinase inhibitors are compounds able to reduce the activity of protein tyrosine kinases. In the present disclosure, considerations set out in respect of “inhibitors” should all be taken as applicable to “protein tyrosine kinase inhibitors”, unless the context requires otherwise.

Kinase inhibitors, such as protein tyrosine kinase inhibitors are frequently used in the pharmaceutical industry, for example in the treatment of inflammation or of cancer. A large number of protein tyrosine kinase inhibitors have been developed in this manner. Protein tyrosine kinase inhibitors of this sort are able to exhibit high selectivity, acceptable toxicity, and other physical, chemical, pharmacological, and biological properties that make them suitable for medical use.

The great degree to which the pharmacological properties of protein tyrosine kinases have been investigated and characterised means that a skilled person will have no difficulty in preparing suitable doses or formulations of these compounds in order to practice the invention, when provided with the information in the present disclosure. For example, presented with a serum concentration of a protein kinase inhibitor (or a particular protein kinase inhibitor) that it is desired to achieve, the skilled person will readily be able to identify corresponding doses of the inhibitor of interest that should be administered to a subject in order to give rise to the requisite concentrations.

A protein tyrosine kinase inhibitor suitable for use in accordance with the invention may be characterised with reference to tyrosine kinase domains that it is able to inhibit. Since the majority of protein tyrosine kinase inhibitors have been developed for use in modifying the activity of kinases within human subjects, their activity is best characterised with respect to inhibition of human kinases. Categorisation in this manner may be useful in grouping together inhibitors suitable for use in the present disclosure, even if when employed in the medical uses or methods of the invention these inhibitors are actually working on bacterial targets within species of the MTBC (rather than inhibiting kinases in host cells). Suitable protein tyrosine kinase inhibitors may also be defined with reference to the substrates phosphorylation of which they are able to inhibit.

Merely by way of example, a suitable protein tyrosine kinase inhibitor for use in the medical uses or methods of treatment of the invention may be selected from the group consisting of: an inhibitor of the tyrosine kinase domain found in the epidermal growth factor receptor (EGFR); an inhibitor of the tyrosine kinase domain found in the platelet derived growth factor-receptor (PDGF-R); an inhibitor of mTOR complex 2 (mTORC2); an inhibitor of the tyrosine kinase domain found in the fibroblast growth factor receptor (FGFR); an inhibitor of src kinase; an inhibitor of an Aurora kinase; an inhibitor of a Janus kinase (JAK); and an inhibitor of a mitogen-activated protein tyrosine kinase (MAPK). In the case of protein tyrosine kinase inhibitor defined with reference to a substrate phosphorylation of which is to be inhibited, a suitable medical use or method of the invention may utilise an protein tyrosine kinase inhibitor that inhibits phosphorylation of signal transducer and activator of transcription 3 (STAT3).

Specific examples of protein tyrosine kinase inhibitors that may be employed in the medical uses or methods of the invention include those selected from the group consisting of: gefitinib; erlotinib; imatinib; everolimus; dovitinib; saracatinib; ENMD-2076; and AT9283.

From the above, it will be appreciated that a suitable tyrosine kinase inhibitor for use in accordance with the invention may inhibit the tyrosine kinase domain found in the epidermal growth factor receptor (EGFR). Gefitinib and erlotinib represent particular examples of such inhibitors that the inventors have found to be effective.

Alternatively, or additionally, a suitable tyrosine kinase inhibitor for use in accordance with the invention may inhibit the tyrosine kinase domain found in the platelet derived growth factor-receptor (PDGF-R). This same tyrosine kinase domain is also found in both abl and c-kit. Imatinib is examples of an inhibitor of this sort that the inventors have found to be effective.

A suitable tyrosine kinase inhibitor for use in accordance with the invention may inhibit the kinase domain found in mTORC2. Everolimus is an example of an inhibitor suitable for use in accordance with this embodiment of the invention.

A suitable tyrosine kinase inhibitor for use in accordance with the invention may inhibit the kinase domain found in the FGFR. Dovitinib is an example of an inhibitor suitable for use in accordance with this embodiment of the invention.

A suitable tyrosine kinase inhibitor for use in accordance with the invention may inhibit the kinase domain of a src kinase. Saracatinib (also known as AZD0530) is an example of an inhibitor suitable for use in accordance with this embodiment of the invention.

A suitable tyrosine kinase inhibitor for use in accordance with the invention may inhibit the kinase domain of an Aurora kinase. ENMD-2076 and AT9283 are both examples of inhibitors suitable for use in accordance with this embodiment of the invention.

A suitable tyrosine kinase inhibitor for use in accordance with the invention may inhibit the kinase domain found in JAK. Cucurbitacin I (JSI-124), ruxolitinib and tofacitinib are examples of inhibitors suitable for use in accordance with this embodiment of the invention.

A suitable tyrosine kinase inhibitor for use in accordance with the invention may inhibit the kinase domain found in MAPK. Ralimetinib is an example of an inhibitor suitable for use in accordance with this embodiment of the invention.

Compounds having suitable inhibitory activity for use in the invention may be identified by virtue of their activity reported in the literature, or by direct assessment of their activity in any suitable method known to those skilled in the art. Merely by way of example, a method of the ninth aspect of the invention represents a suitable embodiment of an assay by which the requisite activity may be identified. Other examples, including assays directly assessing kinase inhibition, will be well known to those working in this field.

As demonstrated in the Examples, gefitinib, erlotinib and imatinib are all protein tyrosine kinase inhibitors that the inventors have shown to be effective in directly inhibiting growth of MTBC species, and thus suitable for use in the treatment of tuberculosis. Each of these compounds has also previously been proposed for use as an anti-tuberculosis medicament for use in HDT. However, none of these protein tyrosine kinase inhibitors have previously been identified as having the capacity to directly inhibit growth of species of the MTBC. The different clinical applications that arise from this different mode of action are discussed extensively herein.

While each of gefitinib, erlotinib and imatinib is suitable for medical use in accordance with the present invention for the treatment of tuberculosis, they also have different profiles of activity, which suit them to somewhat different clinical applications, as described further below.

### Gefitinib

Gefitinib is a tyrosine kinase inhibitor used in chemotherapy. It is administered orally, and is sold under the brand name Iressa®.

Gefitinib is considered to be specific for the tyrosine kinase domain found in the epidermal growth factor receptor (EGFR). The ability of gefitinib to inhibit signaling via the EGFR is able to increase apoptosis among cancer cells in which this receptor is overactive, thus contributing to its chemotherapeutic use.

The second aspect of the present invention provides gefitinib for use in the treatment of tuberculosis, by direct inhibition of growth of a MTBC species. The fifth aspect of the invention provides gefitinib for use in the treatment of tuberculosis, wherein the tuberculosis has been identified as being caused by infection with a particular MTBC species or strain. Corresponding methods of treatment are also provided.

The inventors have found that gefitinib is able to directly inhibit growth of all MTBC species that they have investigated. This offers a considerable clinical benefit, and means that gefitinib may be used as a broad-spectrum agent for treatment of tuberculosis by directly inhibiting MTBC growth. In the context of the present invention, reference to a “broad-spectrum” treatment of this sort should be taken as indicating that gefitinib (or any other such inhibitor demonstrating an equally wide range of growth inhibitory effects) may be used in the treatment of tuberculosis caused by any clinically relevant MTBC species or lineage. Thus, treatment with gefitinib can be expected to be effective without requiring the MTBC species causing a patient's tuberculosis to be identified, and even in the case that such identification has not occurred, or has not proven possible.

As described elsewhere in the present disclosure, the development and increasing prevalence of MDR TB represents a growing challenge in the management of this disease. The broad-spectrum of activity demonstrated by gefitinib in the medical uses and methods of treatment of the invention indicates that this agent may be of particular benefit in the treatment of MDR forms of TB.

### Erlotinib

Erlotinib is also a tyrosine kinase inhibitor used in chemotherapy that is administered orally. Erlotinib is sold under the brand name Tarceva®.

As with gefitinib, erlotinib is considered to be specific for the tyrosine kinase domain found in the EGFR. Inhibition of signaling via the EGFR in cancer cells in which this receptor is overactive is able to increase apoptosis, which underpins erlotinib's use in chemotherapy.

The third aspect of the present invention provides erlotinib for use in the treatment of tuberculosis, by direct inhibition of growth of a MTBC species. The sixth aspect of the invention provides erlotinib for use in the treatment of tuberculosis wherein the tuberculosis has been identified as being caused by particular MTBC species or strains. Corresponding methods of treatment are also provided.

As demonstrated in the Examples below, erlotinib has been shown to directly inhibit growth of Mycobacterium tuberculosis; Mycobacterium africanum; and Mycobacterium bovis. Accordingly, in a suitable example, erlotinib may be used to treat tuberculosis by directly inhibiting growth of a MTBC species selected from the group consisting of: Mycobacterium tuberculosis; Mycobacterium africanum; and Mycobacterium bovis. More specifically, the inventors' results indicate that erlotinib may be used to treat tuberculosis by directly inhibiting growth of a MTBC species selected from the group consisting of: Mycobacterium tuberculosis-lineage2; Mycobacterium tuberculosis-lineage4; Mycobacterium africanum-lineage 5; Mycobacterium africanum-lineage 6; and Mycobacterium bovis. By way of example, the inventors' results indicate that erlotinib may be used to treat tuberculosis by directly inhibiting growth of a MTBC sublineage selected from the group consisting of: Mycobacterium tuberculosis-lineage2 sublineage East Asia; Mycobacterium tuberculosis-lineage4 sublineage Cameroon, Haarlem, LAM or Euro-American; Mycobacterium africanum-lineage5 sublineage West Africa 1; and Mycobacterium africanum-lineage6 sublineage West Africa 2.

The recognition that erlotinib is particularly suitable for use in directly inhibiting growth of these MTBC species enables a patient stratification approach to be taken in which the MTBC species causing a patient's tuberculosis is identified, and this information used to make an informed decision as to a suitable tyrosine kinase inhibitor to be used for treatment. Accordingly, the sixth aspect of the invention provides erlotinib for use in the treatment of tuberculosis, wherein the tuberculosis has been identified as caused by infection with a Mycobacterium tuberculosis complex species selected from the group consisting of: Mycobacterium tuberculosis; and Mycobacterium africanum; and Mycobacterium bovis. In particular the tuberculosis may be identified as caused by infection with a Mycobacterium tuberculosis complex species selected from the group consisting of: Mycobacterium tuberculosis-lineage2; Mycobacterium tuberculosis-lineage4; Mycobacterium africanum-lineage 5; Mycobacterium africanum-lineage 6; and Mycobacterium bovis.

Although erlotinib has previously been suggested as an anti-tuberculosis agent, it has not previously been recognised as being especially effective in respect of Mycobacterium tuberculosis (e.g., Mycobacterium tuberculosis-lineage2; Mycobacterium tuberculosis-lineage4), or Mycobacterium africanum (e.g., Mycobacterium africanum-lineage 5; and Mycobacterium africanum-lineage 6) or Mycobacterium bovis. The inventors' findings thus enable a patient stratification approach to be adopted, in which patients having tuberculosis as a consequence of infection with a MTBC species that responds well to direct inhibition of growth by erlotinib are selected for treatment with this agent. Conversely, the stratification approach provided allows patients infected by MTBC species or lineages that will not respond positively to treatment with erlotinib to be excluded from treatment with this agent, thus avoiding unnecessary incidents of treatment that do not have an opportunity to prove effective.

In view of the above, it will be recognised that patients identified as having tuberculosis caused by Mycobacterium tuberculosis, or Mycobacterium africanum (and particularly those having tuberculosis caused by Mycobacterium tuberculosis-lineage2; Mycobacterium tuberculosis-lineage4; Mycobacterium africanum-lineage 5; or Mycobacterium africanum-lineage 6) or Mycobacterium bovis represent a sub-group of the total set of tuberculosis patients, and that the patients of this sub-group are able to derive particular benefit from treatment with erlotinib in a manner that has not previously been recognised. The treatment of tuberculosis in accordance with this embodiment is suitably by direct inhibition of growth of a MTBC species as considered herein.

### Imatinib Mesylate

Imatinib mesylate is a tyrosine kinase inhibitor sold under the brand names Gleevec® and Glivec®. It is used as a chemotherapeutic agent, and is normally administered orally.

Imatinib mesylate has been reported to be specific for the tyrosine kinase domain found in abl, c-kit and the platelet derived growth factor-receptor (PDGF-R). The ability of imatinib mesylate to inhibit bcr-abl tyrosine kinase activity is believed to underpin its chemotherapeutic use. Treatment with imatinib mesylate is able to increase apoptosis among cancer cells.

References to imatinib in the present specification should be taken as directed to imatinib mesylate, unless the context requires otherwise.

In its fourth aspect, the invention provides imatinib for use in the treatment of tuberculosis, by direct inhibition of growth of a MTBC species. The seventh aspect of the invention provides imatinib for use in the treatment of tuberculosis, wherein the tuberculosis has been identified as caused by infection with particular MTBC species or strains. Corresponding methods of treatment are also provided.

As demonstrated in the Examples below, imatinib has been shown to directly inhibit growth of Mycobacterium tuberculosis, Mycobacterium africanum and Mycobacterium bovis. Accordingly, in a suitable example, imatinib may be used to treat tuberculosis by directly inhibiting growth of a MTBC species selected from the group consisting of: Mycobacterium tuberculosis; Mycobacterium africanum and Mycobacterium bovis. More specifically, the inventors' results indicate that imatinib may be used to treat tuberculosis by directly inhibiting growth of an MTBC species selected from the group consisting of: Mycobacterium tuberculosis-lineage2; Mycobacterium tuberculosis-lineage4; Mycobacterium africanum-lineage5, Mycobacterium africanum-lineage6 and Mycobacterium bovis. By way of example, the inventors' results indicate that imatinib may be used to treat tuberculosis by directly inhibiting growth of a MTBC sublineage selected from the group consisting of: Mycobacterium tuberculosis-lineage2 sublineage East Asia; Mycobacterium tuberculosis-lineage4 sublineage Cameroon, Haarlem, LAM or Euro-American; Mycobacterium africanum-lineage5 sublineage West Africa 1; and Mycobacterium africanum-lineage6 sublineage West Africa 2.

As with erlotinib, this novel finding on the part of the inventors allows a patient stratification approach to be adopted in which the MTBC species causing a patient's tuberculosis is identified, and this information used to make an informed decision as to a suitable tyrosine kinase inhibitor to be used for treatment. This gives rise to the seventh aspect of the invention, which provides imatinib for use in the treatment of tuberculosis, wherein the tuberculosis has been identified as caused by infection with a MTBC species selected from the group consisting of: Mycobacterium tuberculosis-lineage2; Mycobacterium tuberculosis-lineage4; Mycobacterium africanum-lineage5, Mycobacterium africanum-lineage6 and Mycobacterium bovis. While imatinib has previously been suggested as an anti-tuberculosis agent, it has also not previously been recognised that this agent is especially effective in respect of Mycobacterium tuberculosis (and more specifically, Mycobacterium tuberculosis-lineage2 or Mycobacterium tuberculosis-lineage4), Mycobacterium africanum (and more specifically, Mycobacterium africanum-lineage5 or Mycobacterium africanum-lineage6) or Mycobacterium bovis. Accordingly, patients identified as having tuberculosis caused by Mycobacterium tuberculosis, Mycobacterium africanum or Mycobacterium bovis, and particularly those having tuberculosis Mycobacterium tuberculosis-lineage2, Mycobacterium tuberculosis-lineage4, Mycobacterium africanum-lineage5, Mycobacterium africanum-lineage6 or Mycobacterium bovis, represent a sub-group of patients that are able to derive particular benefit from treatment with imatinib in a manner that has not previously been recognised. The treatment of tuberculosis in accordance with this embodiment is suitably by direct inhibition of growth of a MTBC species as considered herein.

### Identification of MTBC Species Causing Tuberculosis

A number of embodiments of the invention involve the identification of an MTBC species responsible for causing a subject's tuberculosis. Treatment with an appropriate agent may be selected (or treatment with an inappropriate agent avoided) on the basis of this identification.

For the purposes of the present disclosure “identification” in this manner may include “direct identification” (for example by typing of the MTBC species using samples taken from the subject) or “indirect identification”, where other information (such as regarding chain of infection or the predominance of certain MTBC species in particular geographical areas) allows the identity of the MTBC species causing the infection to be inferred.

### Medical Uses and Methods of Treatment of the Invention

The first to seventh aspects of the invention each relate to medical uses. Each of these may be referred to as a “medical use of the invention” and references to medical uses of the invention should be taken as applicable to each of these, unless the context requires otherwise.

The eighth aspect of the invention relates to a method of treatment, referred to on occasion as a “method of treatment of the invention”. Generally, disclosures made in this specification with respect to the medical uses of the invention should also be taken as disclosed in respect of the methods of treatment of the invention, and disclosures made in respect of the methods of treatment of the invention should also be taken as applicable to the medical uses.

The medical uses and methods of treatment of the invention are suitably for practice in respect of treatment of tuberculosis in a human subject, though they may also be practiced in respect of other animals with tuberculosis, as required.

### Therapeutically Effective Amounts of Kinase Inhibitors

In the context of the present invention, therapeutically effective amounts of kinase inhibitors, such as tyrosine kinase inhibitors, are amounts that are able to bring about a direct inhibition of a MTBC species, and thereby alleviate a symptom of active tuberculosis or reduce MTBC infection associated with latent tuberculosis.

A suitable therapeutically effective amount of a protein tyrosine kinase inhibitor for use to treat tuberculosis in a method of treatment in accordance with the present invention or in a medical use of the present invention may be different from a dose of the same inhibitor that is required in order to be therapeutically effective as a host-directed therapeutic agent.

Suitably, a therapeutically effective amount of kinase inhibitor suitable for use in a medical use of the invention, or in the methods of treatment of the eighth aspect of the invention, is lower than a dose of the protein tyrosine kinase inhibitor therapeutically effective as a host directed therapeutic agent.

This embodiment of the invention is based upon the inventors having found that a therapeutically effective amount of a protein tyrosine kinase inhibitor for use in accordance with the present invention (which is to say, for use by directly inhibiting growth of a MTBC species) may be lower than a dose of the same inhibitor that is required in order to be therapeutically effective as a host-directed therapeutic agent.

Merely by way of example, a suitable therapeutically effective amount for use in a medical use or method of treatment of the invention may be sufficient to establish a local concentration of the inhibitor that is 200 μM or less, for example, 175 μM or less, 150 μM or less, 125 μM or less, 100 μM or less, 75 μM or less, 50 μM or less, or even 25 μM or less. The dose may be sufficient to establish a local concentration of the inhibitor that is 15 μM or less, 10 μM or less, 5 μM or less, or even 1 μM or less, or 0.5 μM or less.

Merely by way of example, a therapeutically effective amount of a protein tyrosine kinase inhibitor for use in a medical use or method of treatment of the invention may be a dose sufficient to establish in a recipient a serum concentration of the inhibitor that is between about 0.5 μM and about 26M, about 0.5 μM and about 10M, about 0.5 μM and about 1M, about 0.5 μM and about 10000 μM, about 0.5 μM and about 1500 μM, about 0.5 μM and about 750 μM, about 0.5 μM and about 500 μM, about 5 μM and about 250 μM, about 10 μM and about 150 μM, about 20 μM and about 100 μM, about 30 μM and about 90 μM, 40 μM and about 80 μM or about 50 μM and about 70 μM. Suitably, a therapeutically effective amount of an inhibitor for use in a medical use or method of treatment of the invention may be a dose sufficient to establish in a recipient a serum concentration of the inhibitor that is about 50 μM, about 51 μM, about 52 μM, about 53 μM, about 54 μM, about 55 μM, about 56 μM, about 57 μM, about 58 μM, about 59 μM, about 60 μM, about 61 μM, about 62 μM, about 63 μM, about 64 μM, about 65 μM, about 66 μM, about 67 μM, about 68 μM, about 69 μM or about 70 μM.

In a suitable example, a therapeutically effective amount of an inhibitor for use in a medical use or method of treatment of the invention may be a dose sufficient to establish in a recipient a serum concentration of the inhibitor of 200 μM or less, 190 μM or less, 180 μM or less, 170 μM or less, 160 μM or less, 150 μM or less, 140 μM or less, 130 μM or less, 120 μM or less, 110 μM or less, 100 μM or less, 90 μM or less, 85 μM or less, 80 μM or less, 75 μM or less, 70 μM or less, 65 μM or less, 60 μM or less, 55 μM or less, 50 μM or less, 45 μM or less, 40 μM or less, 35 μM 30 μM or less, 25 μM or less, 20 μM or less, 15 μM or less, or less or even 10 μM or less.

Merely by way of example, a therapeutically effective amount of imatinib for use in a medical use or method of treatment of the invention may be a dose sufficient to establish in a recipient a serum concentration of the inhibitor that is between about 0.5 μM and about 26M, about 0.5 μM and about 10M, about 0.5 μM and about 1M, about 0.5 μM and about 10000 μM, about 0.5 μM and about 1500 μM, about 0.5 μM and about 750 μM, about 0.5 μM and about 500 μM, about 5 μM and about 250 μM, about 10 μM and about 150 μM, about 20 μM and about 100 μM, about 30 μM and about 90 μM, 40 μM and about 80 μM or about 50 μM and about 70 μM. Suitably, a therapeutically effective amount of imatinib for use in a medical use or method of treatment of the invention may be a dose sufficient to establish in a recipient a serum concentration of the inhibitor that is about 25 μM, about 30 μM, about 35 μM, about 40 μM, about 45 μM, about 50 μM, about 51 μM, about 52 μM, about 53 μM, about 54 μM, about 55 μM, about 56 μM, about 57 μM, about 58 μM, about 59 μM, about 60 μM, about 61 μM, about 62 μM, about 63 μM, about 64 μM, about 65 μM, about 66 μM, about 67 μM, about 68 μM, about 69 μM, about 70 μM about 75 μM, about 80 μM, about 85 μM, about 90 μM, or about 95 μM.

In a suitable example, a therapeutically effective amount of imatinib for use in a medical use or method of treatment of the invention may be a dose sufficient to establish in a recipient a serum concentration of 200 μM or less, 190 μM or less, 180 μM or less, 170 μM or less, 160 μM or less, 150 μM or less, 140 μM or less, 130 μM or less, 120 μM or less, 110 μM or less, 100 μM or less, 90 μM or less, 85 μM or less, 80 μM or less, 75 μM or less, 70 μM or less, 65 μM or less, 60 μM or less, 55 μM or less, 50 μM or less, 45 μM or less, 40 μM or less, 35 μM or less, 30 μM or less, 25 μM or less, 20 μM or less, 15 μM or less, or less or even 10 μM or less.

In a suitable embodiment, a therapeutically effective amount of an inhibitor for use in a medical use or method of treatment for Mycobacterium tuberculosis-lineage 2 may be a dose sufficient to establish in a recipient a serum concentration that is between about 5 μM and about 75 μM, about 15 μM and about 65 μM, about 25 μM and about 55 μM or about 35 μM and about 45 μM. In a suitable embodiment, a therapeutically effective amount of an inhibitor for use in a medical use or method of treatment for Mycobacterium tuberculosis-lineage 2 may be a dose sufficient to establish in a recipient a serum concentration that is about 30 μM, about 31 μM, about 32 μM, about 33 μM, about 34 μM, about 35 μM, about 36 μM, about 37 μM, about 38 μM, about 39 μM, about 40 μM, about 41 μM, about 42 μM, about 43 μM, about 44 μM, about 45 μM, about 46 μM, about 47 μM, about 48 μM, about 49 μM, about 50 μM, about 51 μM, about 52 μM, about 53 μM, about 54 μM, about 55 μM, about 56 μM, about 57 μM, about 58 μM, about 59 μM, about 60 μM, about 61 μM, about 62 μM, about 63 μM, about 64 μM, or about 65 μM.

In a suitable embodiment, a therapeutically effective amount of an inhibitor for use in a medical use or method of treatment for Mycobacterium tuberculosis-lineage 2 may be a dose sufficient to establish in a recipient a serum concentration of 75 μM or less, 70 μM or less, 65 μM or less, 60 μM or less, 55 μM or less, 50 μM or less, 45 μM or less, 40 μM or less, 35 μM or less, 30 μM or less, 25 μM or less, 20 μM or less, 15 μM or less, 10 μM or less, or even 5 μM or less.

In a suitable embodiment, a therapeutically effective amount of imatinib for use in a medical use or method of treatment for Mycobacterium tuberculosis-lineage 2 may be a dose sufficient to establish in a recipient a serum concentration that is between about 5 μM and about 75 μM, about 15 μM and about 65 μM, about 25 μM and about 55 μM or about 35 μM and about 45 μM. In a suitable embodiment, a therapeutically effective amount of imatinib for use in a medical use or method of treatment for Mycobacterium tuberculosis-lineage 2 may be a dose sufficient to establish in a recipient a serum concentration that is about 30 μM, about 31 μM, about 32 μM, about 33 μM, about 34 μM, about 35 μM, about 36 μM, about 37 μM, about 38 μM, about 39 μM, about 40 μM, about 41 μM, about 42 μM, about 43 μM, about 44 μM, about 45 μM, about 46 μM, about 47 μM, about 48 μM, about 49 μM, about 50 μM, about 51 μM, about 52 μM, about 53 μM, about 54 μM, about 55 μM, about 56 μM, about 57 μM, about 58 μM, about 59 μM, about 60 μM, about 61 μM, about 62 μM, about 63 μM, about 64 μM, or about 65 μM.

In a suitable embodiment, a therapeutically effective amount of imatinib for use in a medical use or method of treatment for Mycobacterium tuberculosis-lineage 2 may be a dose sufficient to establish in a recipient a serum concentration of 75 μM or less, 70 μM or less, 65 μM or less, 60 μM or less, 55 μM or less, 50 μM or less, 45 μM or less, 40 μM or less, 35 μM or less, 30 μM or less, 25 μM or less, 20 μM or less, 15 μM or less, 10 μM or less, or even 5 μM or less.

In a suitable embodiment, a therapeutically effective amount of erlotinib for use in a medical use or method of treatment of the invention may be a dose sufficient to establish in a recipient a serum concentration that is between about 5 μM and about 75 μM, about 5 μM and about 65 μM, about 5 μM and about 55 μM, about 5 μM and about 50 μM, about 5 μM and about 40 μM, about 5 μM and about 30 μM, or about 5 μM and about 20 μM. In a suitable embodiment, a therapeutically effective amount of erlotinib for use in a medical use or method of treatment of the invention may be a dose sufficient to establish in a recipient a serum concentration that is about 20 μM, about 19 μM, about 18 μM, about 17 μM, about 16 μM, about 15 μM, about 14 μM, about 13 μM, about 12 μM, about 11 μM, about 10 μM, about 9 μM, about 8 μM, about 7 μM, about 6 μM, or about 5 μM.

In a suitable embodiment, a therapeutically effective amount of erlotinib for use in a medical use or method of treatment of the invention may be a dose sufficient to establish in a recipient a serum concentration of 20 μM or less, 19 μM or less, 18 μM or less, 17 μM or less, 16 μM or less, 15 μM or less, 14 μM or less, 13 μM or less, 12 μM or less, 11 μM or less, 10 μM or less, 9 μM or less, 8 μM or less, 7 μM or less, 6 μM or less, or even 5 μM or less.

In a suitable embodiment, a therapeutically effective amount of erlotinib for use in a medical use or method of treatment for Mycobacterium tuberculosis-lineage 2 may be a dose sufficient to establish in a recipient a serum concentration that is between about 5 μM and about 75 μM, about 15 μM and about 65 μM, about 25 μM and about 55 μM or about 35 μM and about 45 μM. In a suitable embodiment, a therapeutically effective amount of erlotinib for use in a medical use or method of treatment for Mycobacterium tuberculosis-lineage 2 may be a dose sufficient to establish in a recipient a serum concentration that is between about 5 μM and about 75 μM, about 5 μM and about 65 μM, about 5 μM and about 55 μM, about 5 μM and about 50 μM, about 5 μM and about 40 μM, about 5 μM and about 30 μM, or about 5 μM and about 20 μM. In a suitable embodiment, a therapeutically effective amount of erlotinib for use in a medical use or method of treatment for Mycobacterium tuberculosis-lineage 2 may be a dose sufficient to establish in a recipient a serum concentration that is about 20 μM, about 19 μM, about 18 μM, about 17 μM, about 16 μM, about 15 μM, about 14 μM, about 13 μM, about 12 μM, about 11 μM, about 10 μM, about 9 μM, about 8 μM, about 7 μM, about 6 μM, or about 5 μM.

In a suitable embodiment, a therapeutically effective amount of erlotinib for use in a medical use or method of treatment for Mycobacterium tuberculosis-lineage 2 may be a dose sufficient to establish in a recipient a serum concentration of 20 μM or less, 19 μM or less, 18 μM or less, 17 μM or less, 16 μM or less, 15 μM or less, 14 μM or less, 13 μM or less, 12 μM or less, 11 μM or less, 10 μM or less, 9 μM or less, 8 μM or less, 7 μM or less, 6 μM or less, or even 5 μM or less.

In a suitable embodiment, a therapeutically effective amount of gefitinib for use in a medical use or method of treatment of the invention may be a dose sufficient to establish in a recipient a serum concentration that is between about 5 μM and about 75 μM, about 15 μM and about 65 μM, about 25 μM and about 55 μM or about 35 μM and about 45 μM. In a suitable embodiment, a therapeutically effective amount of gefitinib for use in a medical use or method of treatment of the invention may be a dose sufficient to establish in a recipient a serum concentration that is about 30 μM, about 31 μM, about 32 μM, about 33 μM, about 34 μM, about 35 μM, about 36 μM, about 37 μM, about 38 μM, about 39 μM, about 40 μM, about 41 μM, about 42 μM, about 43 μM, about 44 μM, about 45 μM, about 46 μM, about 47 μM, about 48 μM, about 49 μM, about 50 μM, about 51 μM, about 52 μM, about 53 μM, about 54 μM, about 55 μM, about 56 μM, about 57 μM, about 58 μM, about 59 μM, or about 60 μM.

In a suitable embodiment, a therapeutically effective amount of gefitinib for use in a medical use or method of treatment of the invention may be a dose sufficient to establish in a recipient a serum concentration of 75 μM or less, 70 μM or less, 65 μM or less, 60 μM or less, 55 μM or less, 50 μM or less, 45 μM or less, 40 μM or less, 35 μM or less, 30 μM or less, 25 μM or less, 20 μM or less, 15 μM or less, 10 μM or less, or even 5 μM or less.

In a suitable embodiment, a therapeutically effective amount of gefitinib for use in a medical use or method of treatment for Mycobacterium tuberculosis-lineage 2 may be a dose sufficient to establish in a recipient a serum concentration that is between about 5 μM and about 75 μM, about 15 μM and about 65 μM, about 25 μM and about 60 μM, about 45 μM and about 55 μM, or about 50 μM and about 55 μM. In a suitable embodiment, a therapeutically effective amount of gefitinib for use in a medical use or method of treatment for Mycobacterium tuberculosis-lineage 2 may be a dose sufficient to establish in a recipient a serum concentration that is about 30 μM, about 31 μM, about 32 μM, about 33 μM, about 34 μM, about 35 μM, about 36 μM, about 37 μM, about 38 μM, about 39 μM, about 40 μM, about 41 μM, about 42 μM, about 43 μM, about 44 μM, about 45 μM, about 46 μM, about 47 μM, about 48 μM, about 49 μM, about 50 μM, about 51 μM, about 52 μM, about 53 μM, about 54 μM, about 55 μM, about 56 μM, about 57 μM, about 58 μM, about 59 μM, or about 60 μM.

In a suitable embodiment, a therapeutically effective amount of gefitinib for use in a medical use or method of treatment for Mycobacterium tuberculosis-lineage 2 may be a dose sufficient to establish in a recipient a serum concentration of 75 μM or less, 70 μM or less, 65 μM or less, 60 μM or less, 55 μM or less, 50 μM or less, 45 μM or less, 40 μM or less, 35 μM or less, 30 μM or less, 25 μM or less, 20 μM or less, 15 μM or less, 10 μM or less, or even 5 μM or less.

In a suitable embodiment, a therapeutically effective amount of an inhibitor for use in a medical use or method of treatment for Mycobacterium tuberculosis-lineage 4 may be a dose sufficient to establish in a recipient a serum concentration that is between about 50 μM and about 150 μM, about 60 μM and about 140 μM, about 70 μM and about 130 μM, about 80 μM and about 120 μM or about 90 μM and about 110 μM. In a suitable embodiment, a therapeutically effective amount of an inhibitor for use in a medical use or method of treatment for Mycobacterium tuberculosis-lineage 4 may be a dose sufficient to establish in a recipient a serum concentration that is about 140 μM, about 141 μM, about 142 μM, about 143 μM, about 144 μM, about 145 μM, about 146 μM, about 147 μM, about 148 μM, about 149 μM, about 150 μM, about 151 μM, about 152 μM, about 153 μM, about 154 μM, about 155 μM, about 156 μM, about 157 μM, about 158 μM, about 159 μM or about 160 μM.

In a suitable example, a therapeutically effective amount of an inhibitor for use in a medical use or method of treatment for Mycobacterium tuberculosis-lineage 4 may be a dose sufficient to establish in a recipient a serum concentration of 150 μM or less, 140 μM or less, 130 μM or less, 120 μM or less, 110 μM or less, 100 μM or less, 90 μM or less, 80 μM or less, 70 μM or less, 60 μM or less or even 50 μM or less.

In a suitable embodiment, a therapeutically effective amount of imatinib for use in a medical use or method of treatment for Mycobacterium tuberculosis-lineage 4 may be a dose sufficient to establish in a recipient a serum concentration that is between about 50 μM and about 150 μM, about 60 μM and about 140 μM, about 70 μM and about 130 μM, about 80 μM and about 120 μM or about 90 μM and about 110 μM. In a suitable embodiment, a therapeutically effective amount of imatinib for use in a medical use or method of treatment for Mycobacterium tuberculosis-lineage 4 may be a dose sufficient to establish in a recipient a serum concentration that is about 140 μM, about 141 μM, about 142 μM, about 143 μM, about 144 μM, about 145 μM, about 146 μM, about 147 μM, about 148 μM, about 149 μM, about 150 μM, about 151 μM, about 152 μM, about 153 μM, about 154 μM, about 155 μM, about 156 μM, about 157 μM, about 158 μM, about 159 μM or about 160 μM.

In a suitable example, a therapeutically effective amount of imatinib for use in a medical use or method of treatment for Mycobacterium tuberculosis-lineage 4 may be a dose sufficient to establish in a recipient a serum concentration of 150 μM or less, 140 μM or less, 130 μM or less, 120 μM or less, 110 μM or less, 100 μM or less, 90 μM or less, 80 μM or less, 70 μM or less, 60 μM or less or even 50 μM or less.

In a suitable embodiment, a therapeutically effective amount of an inhibitor for use in a medical use or method of treatment for Mycobacterium bovis may be a dose sufficient to establish in a recipient a serum concentration that is between about 0.5 μM and about 10 μM, about 1 μM and about 9 μM, about 1 μM and about 3 μM, about 2 μM and about 8 μM, about 3 μM and about 7 μM or about 4 μM and about 6 μM. In a suitable embodiment, a therapeutically effective amount of an inhibitor for use in a medical use or method of treatment for Mycobacterium bovis may be a dose sufficient to establish in a recipient a serum concentration that is about 0.5 μM, about 1 μM, about 2 μM, about 3 μM, about 4 μM, about 5 μM, about 6 μM, about 7 μM, about 8 μM, about 9 μM, or about 10 μM.

In a suitable example, a therapeutically effective amount of an inhibitor for use in a medical use or method of treatment for Mycobacterium bovis may be a dose sufficient to establish in a recipient a serum concentration of 10 μM or less, 9 μM or less, 8 μM or less, 7 μM or less, 6 μM or less, 5 μM or less, 4 μM or less, 3 μM or less, 2 μM or less, 1 μM or less or even 0.5 μM or less.

In a suitable embodiment, a therapeutically effective amount of imatinib for use in a medical use or method of treatment for Mycobacterium bovis may be a dose sufficient to establish in a recipient a serum concentration that is between about 0.5 μM and about 10 μM, about 1 μM and about 9 μM, about 2 μM and about 8 μM, about 3 μM and about 7 μM or about 4 μM and about 6 μM. In a suitable embodiment, a therapeutically effective amount of imatinib for use in a medical use or method of treatment for Mycobacterium bovis may be a dose sufficient to establish in a recipient a serum concentration that is about 0.5 μM, about 1 μM, about 2 μM, about 3 μM, about 4 μM, about 5 μM, about 6 μM, about 7 μM, about 8 μM, about 9 μM, or about 10 μM.

In a suitable example, a therapeutically effective amount of imatinib for use in a medical use or method of treatment for Mycobacterium bovis may be a dose sufficient to establish in a recipient a serum concentration of 10 μM or less, 9 μM or less, 8 μM or less, 7 μM or less, 6 μM or less, 5 μM or less, 4 μM or less, 3 μM or less, 2 μM or less, 1 μM or less or even 0.5 μM or less.

In a suitable embodiment, a therapeutically effective amount of an inhibitor for use in a medical use or method of treatment for Mycobacterium africanum-lineage 5 may be a dose sufficient to establish in a recipient a serum concentration that is between about 40 μM and about 60 μM, about 40 μM and about 50 μM, about 50 μM and about 60 μM or about 45 μM and about 55 μM. In a suitable embodiment, a therapeutically effective amount of an inhibitor for use in a medical use or method of treatment for Mycobacterium africanum-lineage 5 may be a dose sufficient to establish in a recipient a serum concentration that is about 40 μM, about 41 μM, about 42 μM, about 43 μM, about 44 μM, about 45 μM, about 46 μM, about 47 μM, about 48 μM, about 49 μM, about 50 μM, about 51 μM, about 52 μM, about 53 μM, about 54 μM, about 55 μM, about 56 μM, about 57 μM, about 58 μM, about 59 μM, or about 60 μM.

In a suitable example, a therapeutically effective amount of an inhibitor for use in a medical use or method of treatment for Mycobacterium africanum-lineage 5 may be a dose sufficient to establish in a recipient a serum concentration of 60 μM or less, 55 μM or less, 50 μM or less, 45 μM or less, 40 μM or less, 35 μM or less, 30 μM or less, 25 μM or less, 20 μM or less, 15 μM or less, 10 μM or less, or even 5 μM or less.

In a suitable embodiment, a therapeutically effective amount of imatinib for use in a medical use or method of treatment for Mycobacterium africanum-lineage 5 may be a dose sufficient to establish in a recipient a serum concentration that is between about 40 μM and about 60 μM, about 40 μM and about 50 μM, about 50 μM and about 60 μM or about 45 μM and about 55 μM. In a suitable embodiment, a therapeutically effective amount of imatinib for use in a medical use or method of treatment for Mycobacterium africanum-lineage 5 may be a dose sufficient to establish in a recipient a serum concentration that is about 40 μM, about 41 μM, about 42 μM, about 43 μM, about 44 μM, about 45 μM, about 46 μM, about 47 μM, about 48 μM, about 49 μM, about 50 μM, about 51 μM, about 52 μM, about 53 μM, about 54 μM, about 55 μM, about 56 μM, about 57 μM, about 58 μM, about 59 μM, or about 60 μM.

In a suitable example, a therapeutically effective amount of imatinib for use in a medical use or method of treatment for Mycobacterium africanum-lineage 5 may be a dose sufficient to establish in a recipient a serum concentration of 60 μM or less, 55 μM or less, 50 μM or less, 45 μM or less, 40 μM or less, 35 μM or less, 30 μM or less, 25 μM or less, 20 μM or less, 15 μM or less, 10 μM or less, or even 5 μM or less.

In a suitable embodiment, a therapeutically effective amount of an inhibitor for use in a medical use or method of treatment for Mycobacterium africanum-lineage 6 may be a dose sufficient to establish in a recipient a serum concentration that is between about 0.5 μM and about 1.5M, about 0.5M and about 1M, about 0.5 μM and about 10000 μM, about 0.5 μM and about 1500 μM, about 0.5 μM and about 750 μM, about 0.5 μM and about 500 μM, about 5 μM and about 250 μM, about 10 μM and about 200 μM, about 20 μM and about 150 μM, about 30 μM and about 1200 μM, 40 μM and about 110 μM, about 50 μM and about 100 μM or about 80 μM and about 100 μM. In a suitable embodiment, a therapeutically effective amount of an inhibitor for use in a medical use or method of treatment for Mycobacterium africanum-lineage 6 may be a dose sufficient to establish in a recipient a serum concentration that is about 80 μM, about 81 μM, about 82 μM, about 83 μM, about 84 μM, about 85 μM, about 86 μM, about 87 μM, about 88 μM, about 89 μM, about 90 μM, about 91 μM, about 92 μM, about 93 μM, about 94 μM, about 95 μM, about 96 μM, about 97 μM, about 98 μM, about 99 μM, or about 100 μM.

In a suitable example, a therapeutically effective amount of an inhibitor for use in a medical use or method of treatment for Mycobacterium africanum-lineage 6 may be a dose sufficient to establish in a recipient a serum concentration of 100 μM or less, 95 μM or less, 90 μM or less, 85 μM or less or even 80 μM or less.

In a suitable embodiment, a therapeutically effective amount of imatinib for use in a medical use or method of treatment for Mycobacterium africanum-lineage 6 may be a dose sufficient to establish in a recipient a serum concentration that is between about 0.5 μM and about 1.5M, about 0.5 μM and about 1M, about 0.5 μM and about 10000 μM, about 0.5 μM and about 1500 μM, about 0.5 μM and about 750 μM, about 0.5 μM and about 500 μM, about 5 μM and about 250 μM, about 10 μM and about 150 μM, about 20 μM and about 100 μM, about 30 μM and about 90 μM, 40 μM and about 80 μM or about 50 μM and about 70 μM. In a suitable embodiment, a therapeutically effective amount of an imatinib for use in a medical use or method of treatment for Mycobacterium africanum-lineage 6 may be a dose sufficient to establish in a recipient a serum concentration that is about 80 μM, about 81 μM, about 82 μM, about 83 μM, about 84 μM, about 85 μM, about 86 μM, about 87 μM, about 88 μM, about 89 μM, about 90 μM, about 91 μM, about 92 μM, about 93 μM, about 94 μM, about 95 μM, about 96 μM, about 97 μM, about 98 μM, about 99 μM, or about 100 μM.

In a suitable example, a therapeutically effective amount of imatinib for use in a medical use or method of treatment for Mycobacterium africanum-lineage 6 may be a dose sufficient to establish in a recipient a serum concentration of 100 μM or less, 95 μM or less, 90 μM or less, 85 μM or less or even 80 μM or less.

The ability to gain therapeutic benefit from lower doses of protein tyrosine kinase inhibitors confers notable benefits in terms of the medical uses and methods of treatment of the invention. It is recognised that protein tyrosine kinase inhibitors are powerful pharmaceutical agents, and that their cause may be associated with considerable toxicity in contexts such as chemotherapy for cancer. In view of this, the identification that such compounds can be used at lower concentrations, while still providing effective treatment for tuberculosis by direct inhibition of the growth of MTBC species, is clearly advantageous.

### Routes of Administration

A medical use or method of treatment of the invention may involve provision of a therapeutically effective amount of a protein tyrosine kinase inhibitor to a subject requiring such treatment by any suitable means. Suitably the inhibitor may be provided by oral administration. Each of gefitinib, erlotinib and imatinib are known to be suitable for oral administration.

Provision of orally administered agents is generally associated with good rates of patient compliance. The ability of the medical uses and methods of treatment of the invention to achieve effective treatment of TB without the need for injectable drugs may be of particular benefit in the context of MDR TB, where injections are currently frequently required.

### Formulation of Pharmaceutical Compositions for the Medical Uses and Methods of Treatment of the Invention

A therapeutically effective amount of a protein tyrosine kinase inhibitor will be provided to a subject by means of a pharmaceutical composition. Examples of suitable formulations by which inhibitors, such as gefitinib, erlotinib or imatinib, may be administered are well known to those skilled in the art. Such formulations may be modified, as required, in light of the doses of protein tyrosine kinase inhibitors that have been newly identified as therapeutically effective in the present disclosure.

### Methods for Investigating Anti-MTBC Abilities of Protein Tyrosine Kinase Inhibitors

As referred to elsewhere in this specification, the inventors believe that a lack of sensitivity in the colony forming unit (CFU) assay most commonly used in this field has contributed to the fact that the ability of protein tyrosine kinase inhibitors to directly inhibit growth of MTBC species has not been noted before.

Furthermore, previous experimental methods for investigating anti-tuberculosis agents have focused on “laboratory” strains, rather than clinically relevant species and lineages.

The inventors methods of the ninth and tenth aspects of the invention (respectively directed to a method of determining the ability of a protein tyrosine kinase inhibitor to inhibit growth of a Mycobacterium tuberculosis complex species, and a method of identifying a protein tyrosine kinase inhibitor as being potentially effective in the treatment of tuberculosis by direct inhibition of a Mycobacterium tuberculosis complex species) seek to overcome these problems.

Both methods employ clinically relevant MTBC species. These may be selected from the group consisting of: Mycobacterium tuberculosis-lineage2; Mycobacterium tuberculosis-lineage4; Mycobacterium africanum-lineage5; Mycobacterium africanum-lineage6; and Mycobacterium bovis.

Both methods also involve assessing growth of the MTBC species by means of an assay selected from the group consisting of: an assay that directly measures bacterial growth; an assay that measures bacterial growth via accumulation of a reporter; and an assay that measures actively multiplying bacteria.

The assay that directly measures bacterial growth may suitably assess absorbance or transmittance of light through a sample comprising the MTBC species. Suitably it may assess absorbance or transmittance of light at a wavelength of approximately 600 nm.

The assay that measures bacterial growth via accumulation of a reporter and assay that measures actively multiplying bacteria may both make use of modified bacteria, for example modified to express a reporter or a component of a luminescence reaction system.

Suitably the reporter is a reporter protein. Examples of such a reporter protein include those selected from the group consisting of: a fluorescent reporter protein; and a colorigenic reporter protein. Fluorescent reporter proteins suitable for use in such methods include those selected from the group consisting of: green fluorescent protein (GFP); Mcherry; and dsRed2.

The assay that measures actively multiplying bacteria may assesses luminescence of the bacteria, for example in the case of bacteria modified to express luciferase.

The methods developed by the inventors are more sensitive than those of the prior art (based on measurement of CFUs), and also allow data to be generated faster and more robustly than in prior art methods. MTBC species are known to have very slow growth rates. Prior art methods may take three to four weeks to generate suitable results, whereas the methods of the invention are able to highlight direct growth inhibition by test compounds in as little as five days.

Given the lack of sensitivity of CFU-based assays, and their slow time to produce effective readouts, a suitable embodiment of a method of either the ninth or tenth aspect of the invention may not include a measurement of colony formation by the Mycobacterium tuberculosis complex species.

The invention will now be further described, with reference to the following Examples.

### EXAMPLES

### Example 1

Background: The rise of multidrug-resistant tuberculosis (TB) and poor treatment outcomes of some drug-susceptible TB mandate novel treatment approaches such as host-directed therapeutics (HDTs). However, the tools to evaluate their efficacy and determine the impact of host and Mycobacterium tuberculosis complex (MTBC) genetic diversity is lacking. This study sought to address this gap to better inform the development of HDTs for TB.

Methods: Clinical isolates representing major MTBC lineages circulating in Africa were transfected with reporter-gene-tagged plasmids and used to assess HDTs' (tyrosine kinase inhibitors) direct inhibition in-vitro using a SpectraMax i3x measuring bacilli growth. Standard colony-forming unit (CFU), together with absorbance, fluorescent, and luminescent readouts, were analysed using a mathematical model based on those used in econometrics to resolve a “composite baseline” against which drugs can be tested accurately and reproducibly and which can predict growth outcomes and drug sensitivity to HDTs.

Results: The growth pattern of Mtb-lineage2, Mtb-lineage4, Maf-lineage5, Maf-lineage6 and M. bovis was significantly different and detectable with all readouts. However, only the fluorescent, luminescent and absorbance readouts were suitable for testing drugs' effect on the bacteria. HDTs, including those in clinical trials for TB such as Gleevec/Imatinib, directly restricted the growth of Mtb-lineage2 and M. bovis but not Mtb-lineage4, and Maf lineages, whereas others like Gefitinib significantly inhibited all the tested MTBC lineages.

Conclusion: We show for the first-time tyrosine kinase inhibitors HDTs' direct inhibition of MTBC lineages in-vitro. The direct inhibition varies with the type of HDT drugs and MTBC lineages. This direct inhibition pathway should be accounted for in selecting efficacious HDTs for all MTBC lineages.

### Example 2

### Introduction

Tuberculosis, caused by members of the Mycobacterium tuberculosis complex (MTBC), claimed 1.4 million lives and caused 10 million new cases worldwide in 2020 (WHO report 2021). TB remains a severe public health threat globally despite the wide use of antibiotic drugs developed over five decades ago. The treatment of drug-susceptible TB takes six months. It uses four antibiotics, including two months with isoniazid (INH), rifampicin (RMP), ethambutol (EMB) and pyrazinamide (PZA), followed by four months with INH and RMP. This lengthy treatment promotes the development of multidrug-resistant TB (MDR TB) associated with poor compliance. MDR TB treatment is more complex, lasting up to 24 months, and uses toxic drugs, including injectables. Thus, shortening TB treatment can improve compliance and significantly reduce the development of MDR TB. This goal requires innovative therapeutics such as adjunctive treatment with drugs targeting patients' immunity, so-called host-directed therapeutics (or HDTs). HDTs can augment anti-TB immunity, reduce immunopathology, enhance the therapeutic effect of anti-bacillary medicines, and reduce the drug pressure favouring resistance to develop (Korbee, Heemskerk et al. 2018). Since HDTs mainly target the host rather than the bacteria, they can shorten the length of both drug-susceptible and MDR TB treatment Fields (Chandra, Rajmani et al. 2016, Gehre, Otu et al. 2016, Korbee, Heemskerk et al. 2018).

Both host and bacteria factors influence TB treatment outcomes. Among bacteria factors, lineages diversity within the MTBC has often been overlooked in the field (Coussens, Wilkinson et al. 2013). However, there is an extensive diversity among MTBC lineages causing TB human; we, we now count nine phylogeographically diversed MTBC lineages (Coscolla et al, 2021). emerging literature demonstrates that MTBC lineages have different capacities to develop drug resistance Fields (Ford, Shah et al. 2013, Gygli, Borrell et al. 2017, Hicks, Yang et al. 2018). Drug-resistant conferring mutations are geographically diverse and are associated with MTBC lineages phylogeography (Hicks, Yang et al. 2018). Moreover, response to the standard TB treatment regimen differs with patients infecting MTBC lineages (Tientcheu, Maertzdorf et al. 2015, Tientcheu, Haks et al. 2016, Diarra, Kone et al. 2018). It becomes essential to account for MTBC lineages' diversity in the development of HDTs.

Most studies have focused on evaluating HDTs activities on intracellular bacteria using macrophage cell lines and primary human and animal-derived macrophages, ignoring their direct action on MTBC (Napier, Rafi et al. 2011, Stanley, Barczak et al. 2014, Napier, Norris et al. 2015, Sogi, Lien et al. 2017). Indeed, MTBC lineages express different protein tyrosine kinases that have critical functions for the bacteria metabolism and can be targeted by protein tyrosine kinase inhibitors (Xu, Wang et al. 2017, Yimer, Kalayou et al. 2020). Previous studies have shown that protein tyrosine kinase inhibitors, including some classes of HDTs drugs under clinical trial, have a direct anti-TB activity (Xu, Wang et al. 2017, Ashley, Hernandez et al. 2020). However, the pool of HDTs candidates tested is limited, and their effects on clinical MTBC lineages have not been studied.

The development of stable reporter-gene-tagged plasmids that can be transfected into clinical MTBC isolates has a significant impact in the high throughput testing of HDT candidates on clinical MTBC lineages. The available technology has relied on the cumbersome colony-forming counts (CFU) method. We present the direct in-vitro activity of HDTs on clinical MTBC lineages representing the major circulating strains in Africa. Our results show that HDTs direct activity on bacilli varies with the drug type and the MTBC lineages. This quantifiable HDTs activity, in addition to the intracellular growth restriction, should be factored in and accounted for in the selection of efficacious HDTs. This approach is fundamental in regions where different MTBC lineages causing TB are co-prevalent.

### Materials and Methods

### Clinical M. Tuberculosis Complex Lineages Stock Generation for Drugs Test

To test the direct effect of host-directed therapeutic (HDTs) drugs on Mycobacterium tuberculosis complex (MTBC), we selected prevalent clinical lineages among West African TB patients. These include M. tuberculosis lineage4, M. tuberculosis lineage2, M. africanum lineage5, M. africanum lineage6 and M. bovis that were transfected with (pMV306Dlhsp+LuxG13, Addgene plasmid #49999), (pBS-Int, Addgene plasmid #50000) and (pGFPHYG2, Addgene plasmid #30173) to confer luminescence and green fluorescence protein (GFP) expression to the bacilli (Andreu, Zelmer et al. 2013). Frozen bacteria aliquots were thawed at room temperature, then subcultured in 15 mL Middlebrook 7H9 medium (Sigma-Aldrich) selective culture media supplemented with 10% albumin-dextrose-catalase (ADC Becton Dickinson, BD) and 0.05% Tween-80 (Sigma-Aldrich). Kanamycin (50 μg/ml) and hygromycin (50 μg/ml) antibiotics (Sigma-Aldrich) were added to allow only the growth of bacteria carrying resistant plasmids. The bacteria cultures were grown at 37° C. with shaking at 70 rpm and monitored by measuring luminescence every 24 hours using either the single tube luminometer (Berthold Detection System) or the SpectraMax i3x (Molecular Devices) until they reached the log phase. The cultures were then top-up with 50 mL of 7H9+10% ADC and grown again in the incubator until the log phase was reached. The bacteria cultures were then harvested with 15% final glycerol concentration, and aliquots of 1 mL were stored in a −80° C. freezer until their use.

### Host-Directed Therapy (HDT) Drugs Preparation

The selected candidate HDT drugs for TB include those that are proposed for clinical trials for active TB Gefitinib and Erlotinib (Selleck chemicals LLC), Cucurbitacin I (Sigma-Aldrich). Those that are undergoing clinical trial Imatinib Mesylate (https://clinicaltrials.gov/ct2/show/results/NCT03891901) and Pravastatin Sodium (https://clinicaltrials.gov/ct2/show/NCT03456102) (Selleck chemicals LLC), and those that have completed clinical trial Auranofin and Everolimus (Sigma-Aldrich) (Wallis et al., 2021) were prepared by dissolving the salts into dimethyl sulfoxide/water DMSO/H₂O solution to make a stock concentration of 5 mM and then stored at −20° C. Control drugs Rifampicin and Isoniazid were prepared to a stock concentration of 83 μg/mL and 8.3 μg/ml, respectively. The concentration of DMSO in the bacteria culture did not exceed 1% final concentration.

### Direct HDT Drugs Activity on MTBC Lineages Test Assay

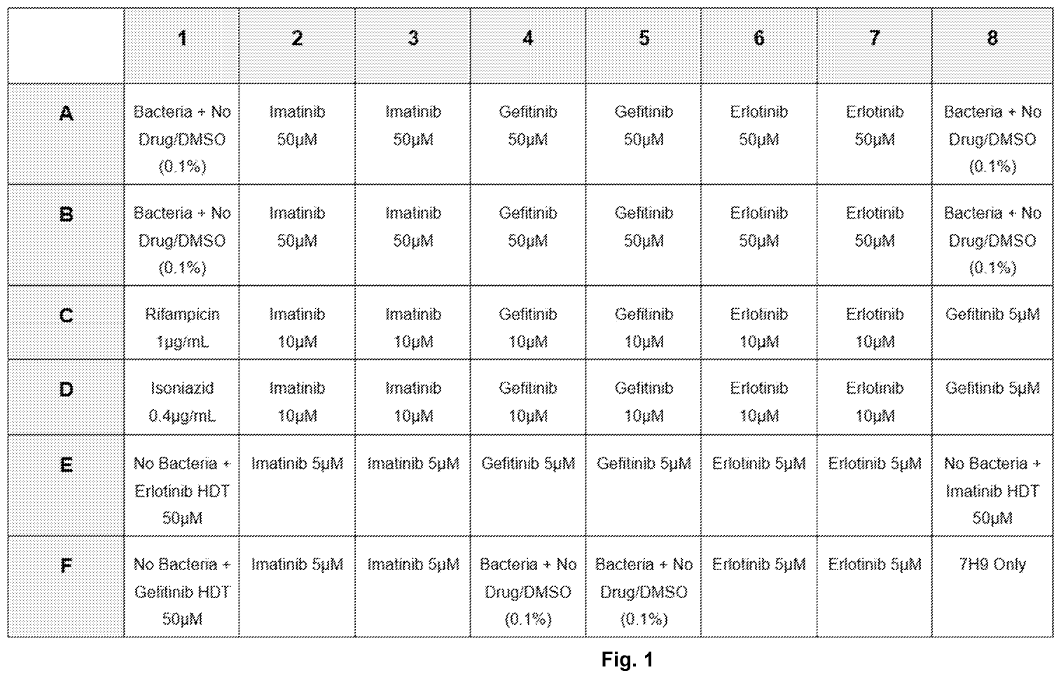

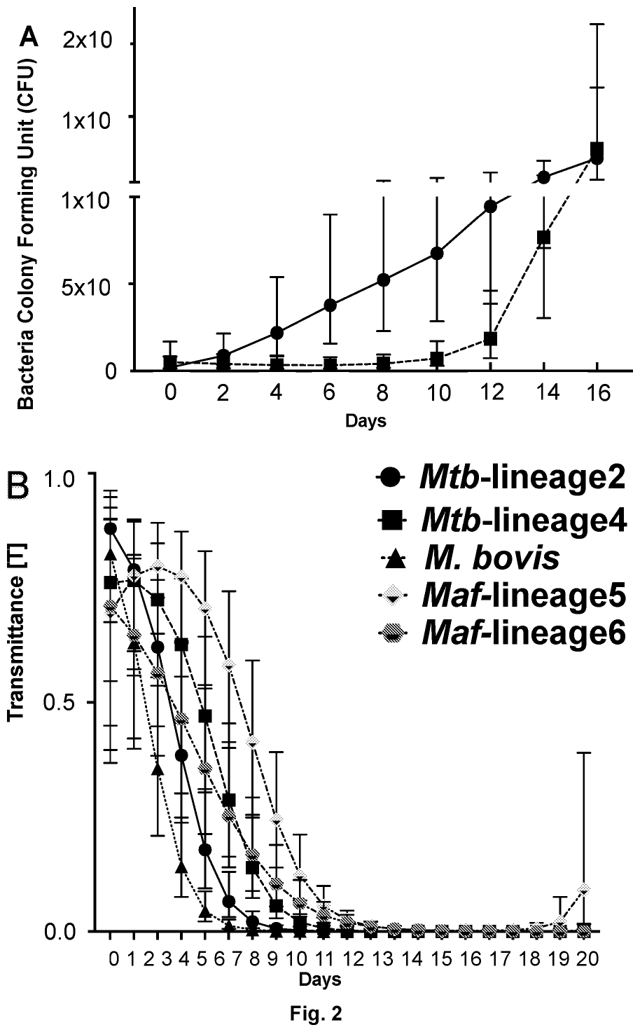

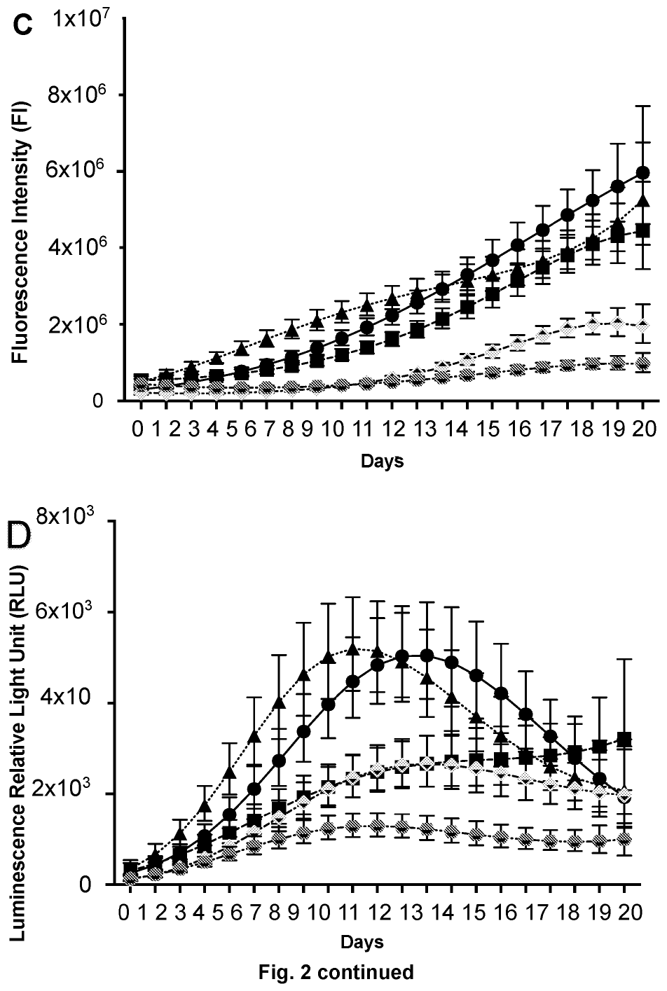

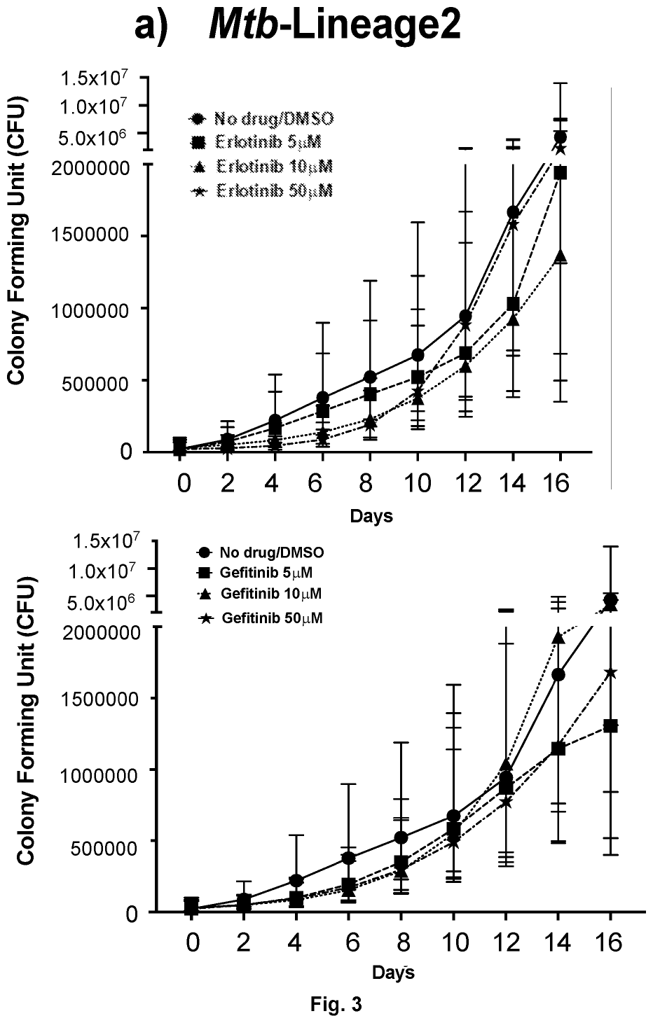

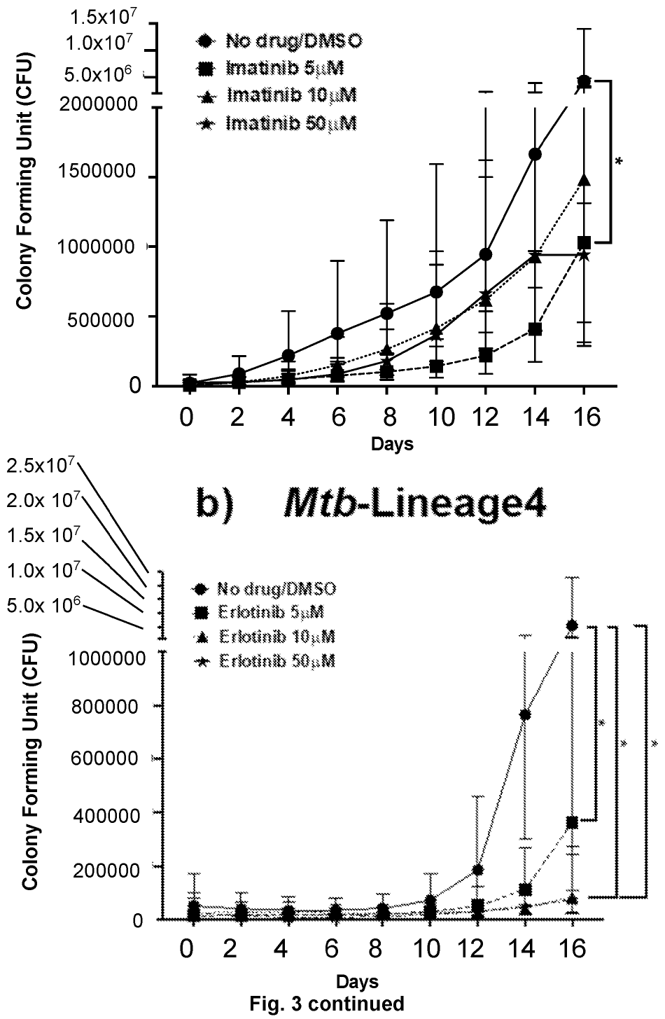

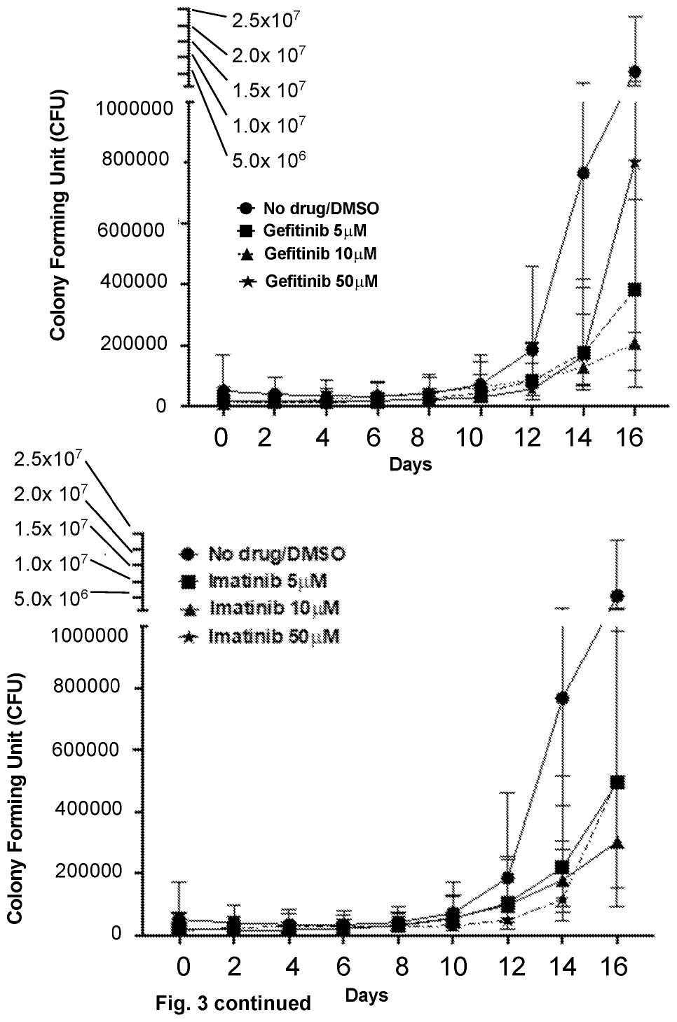

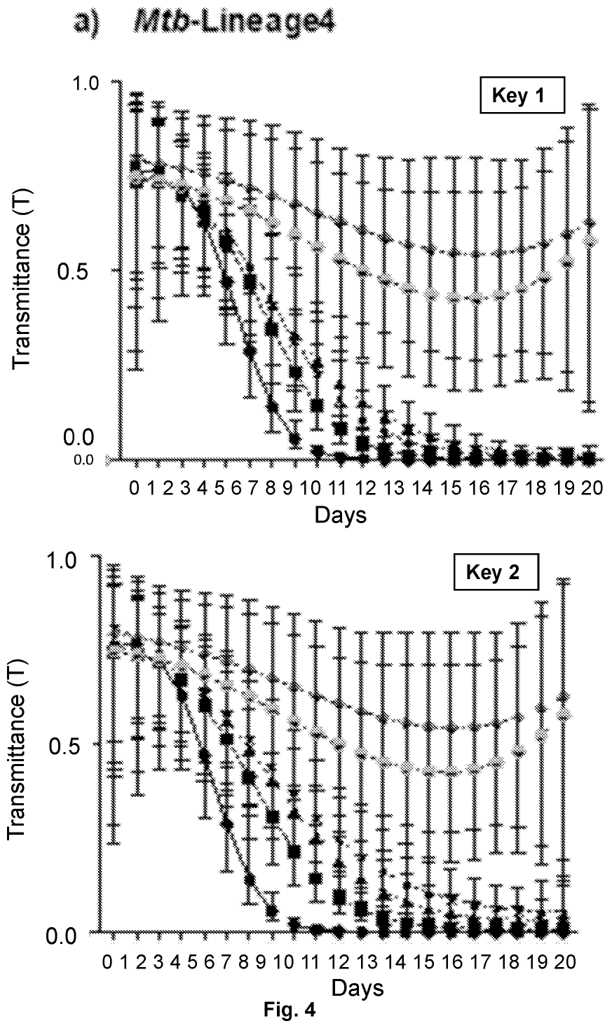

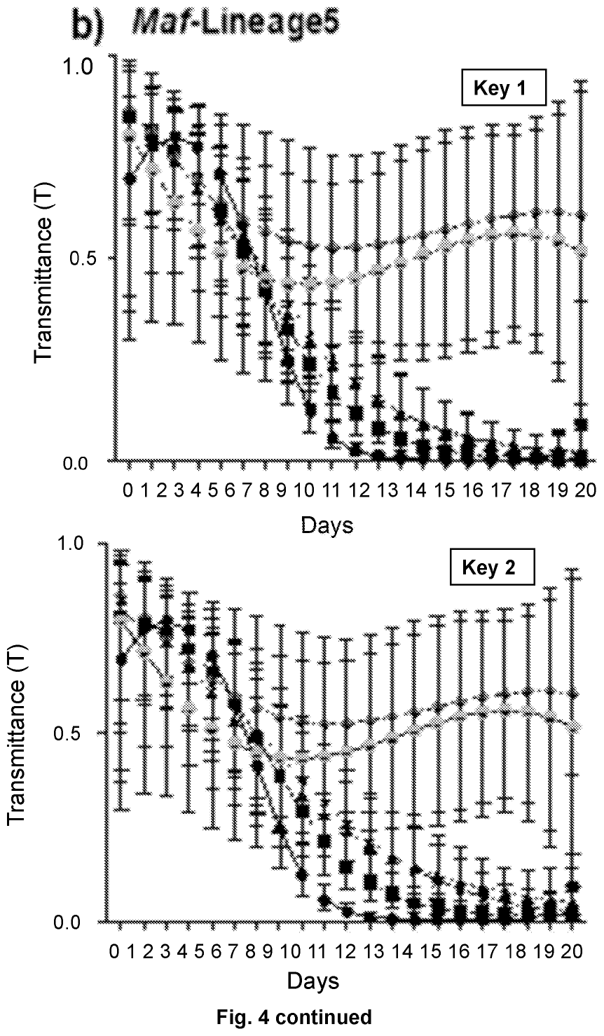

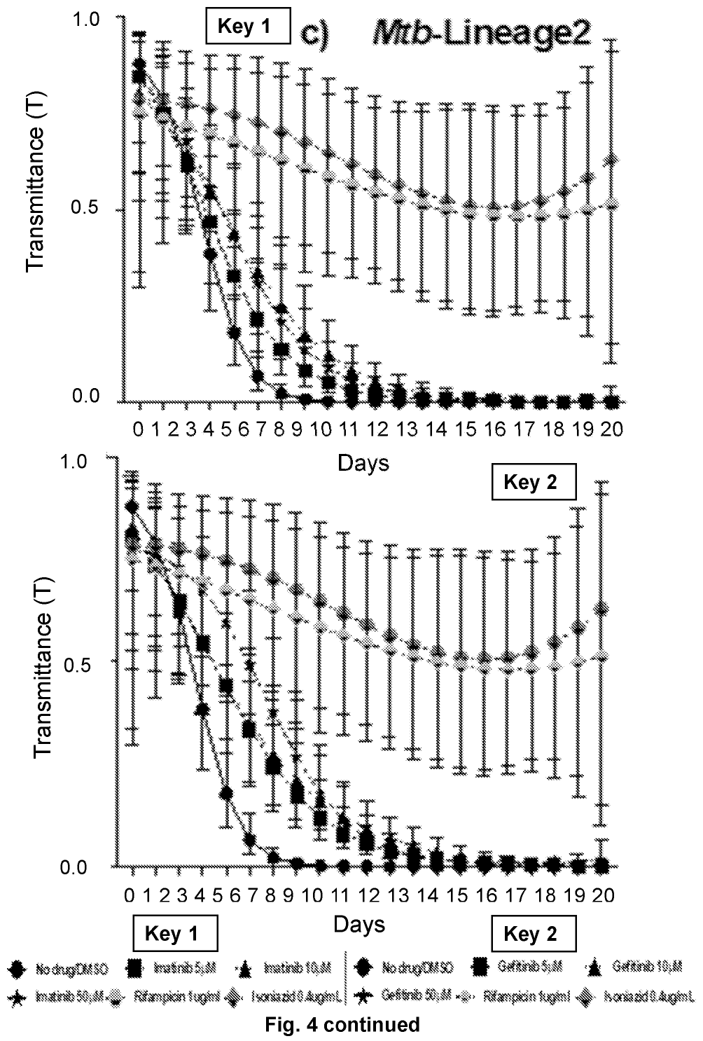

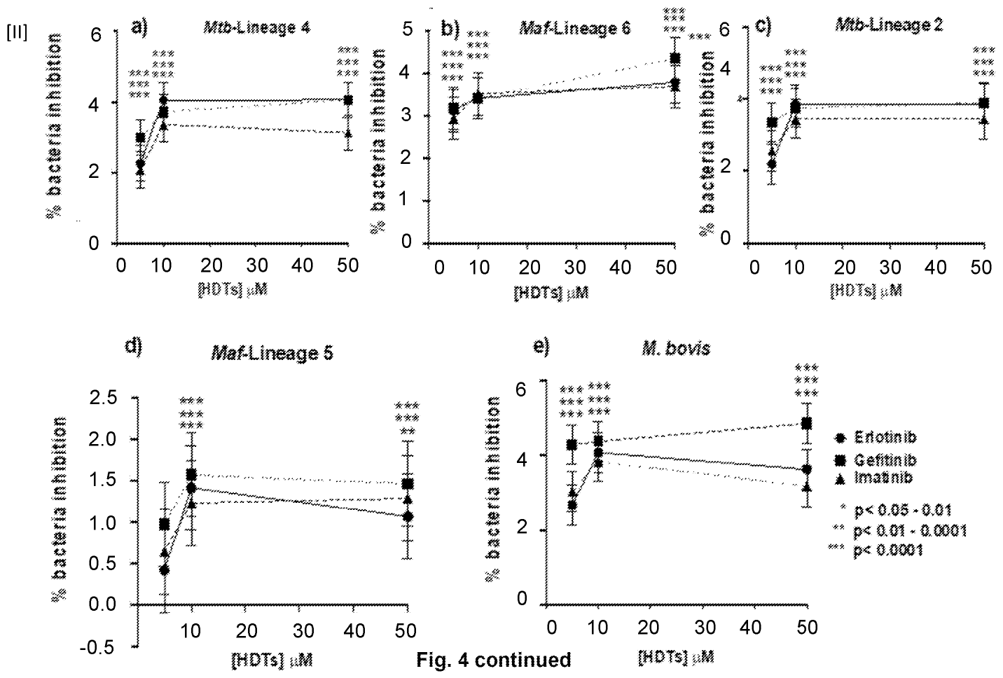

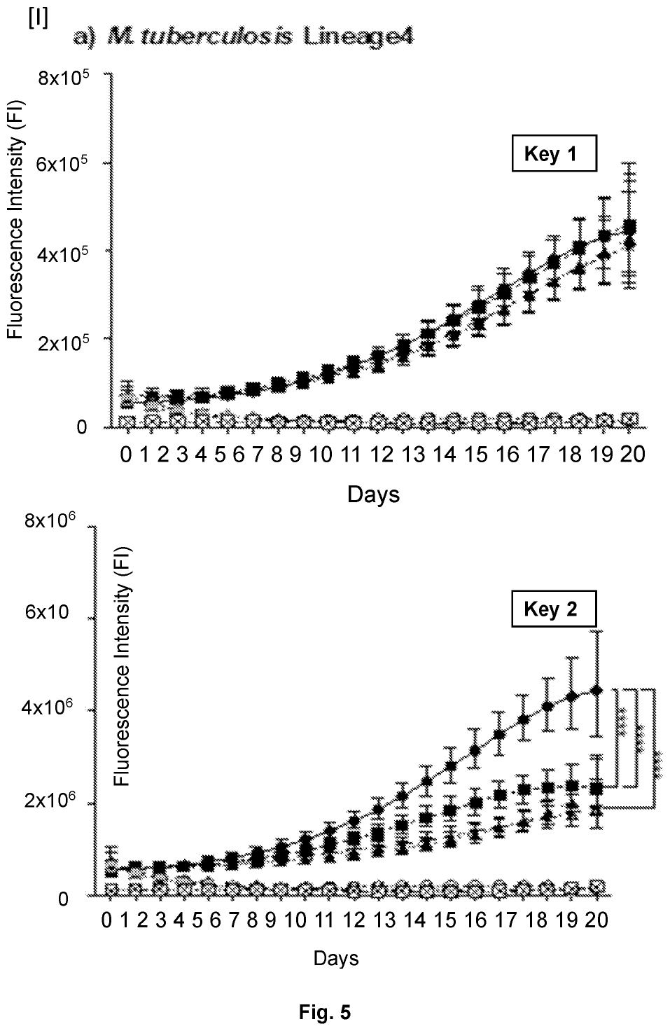

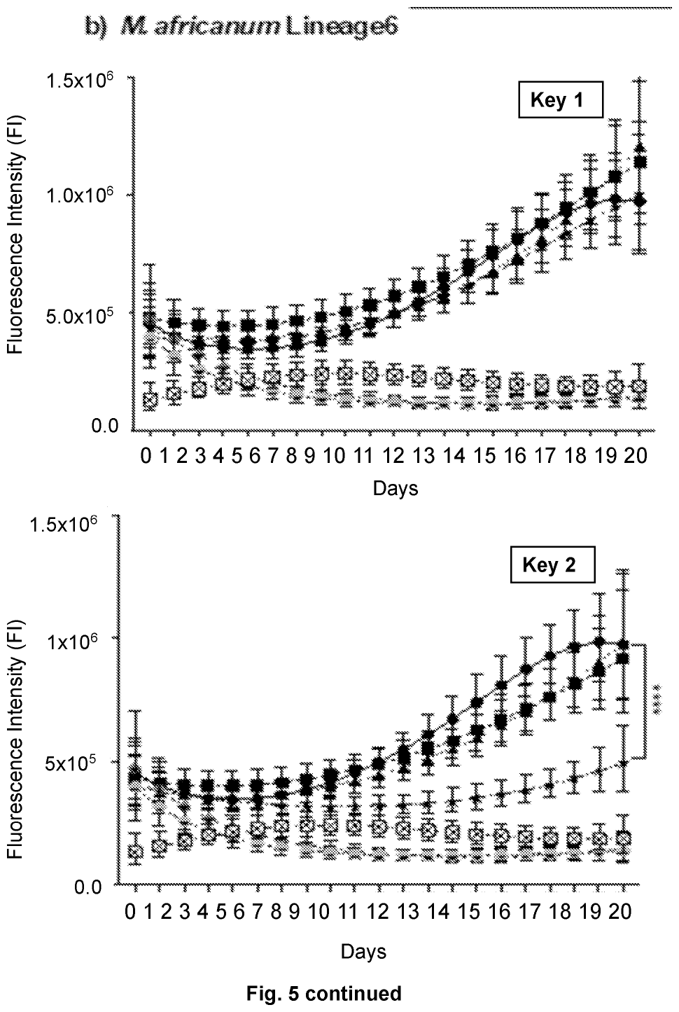

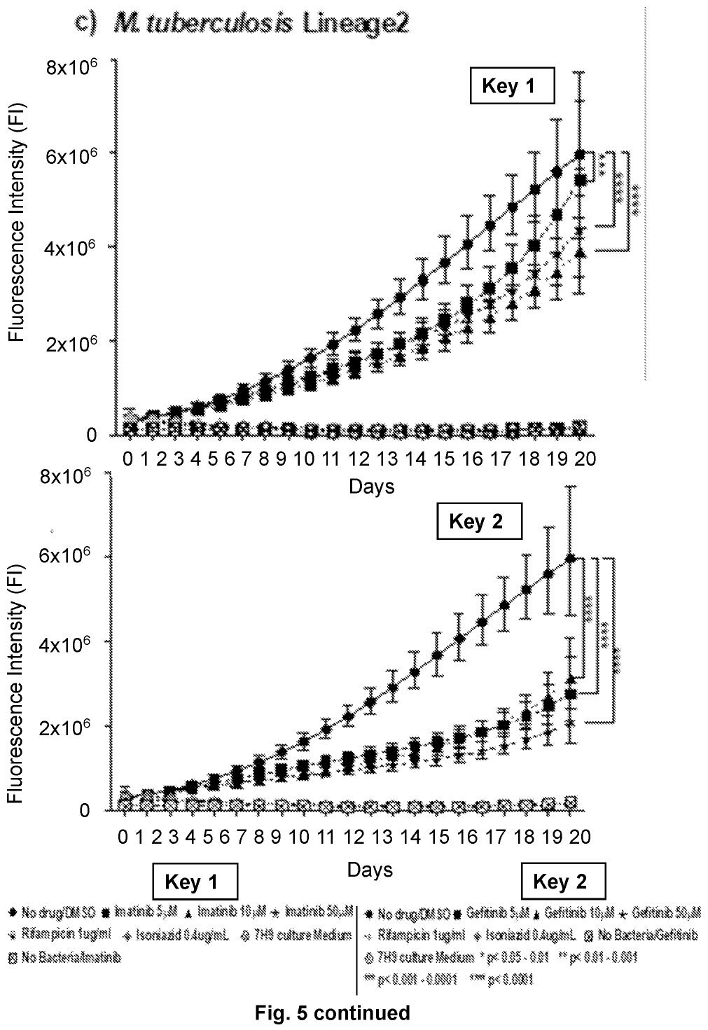

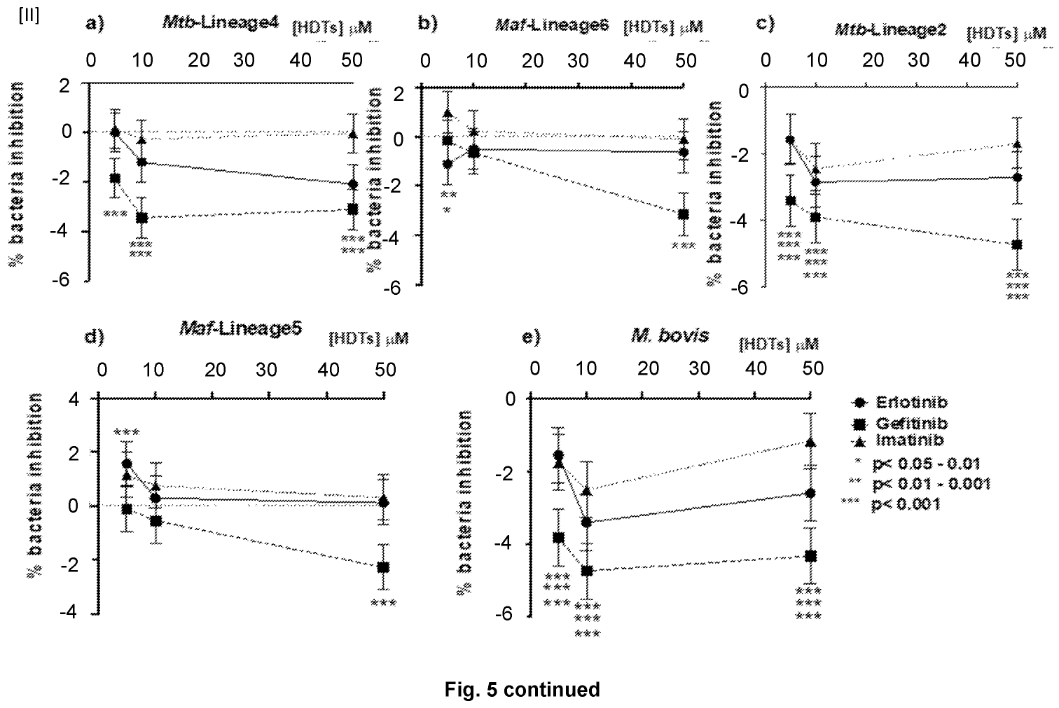

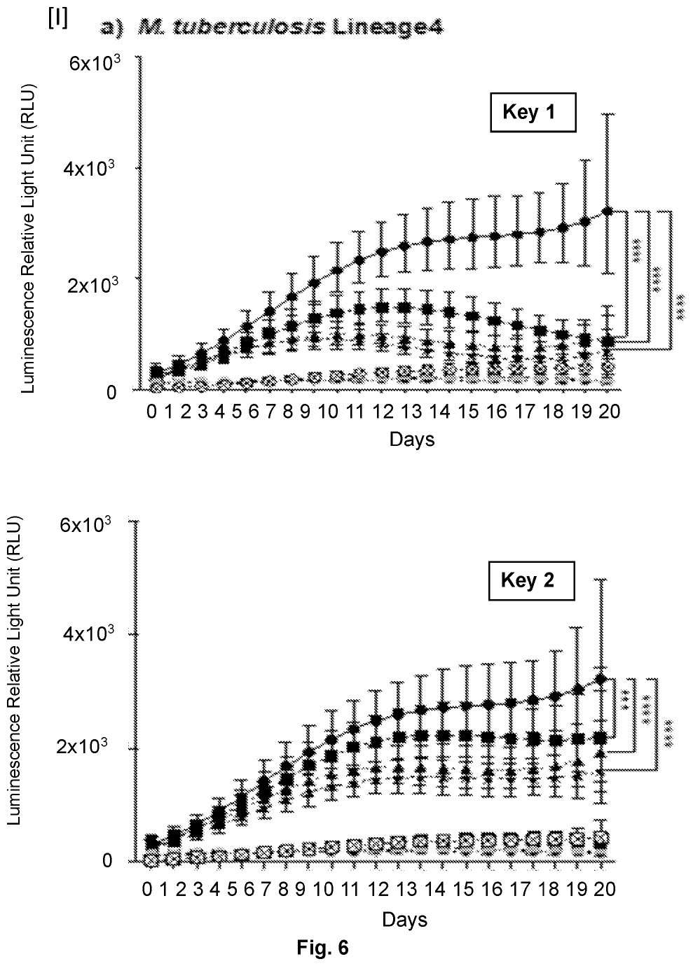

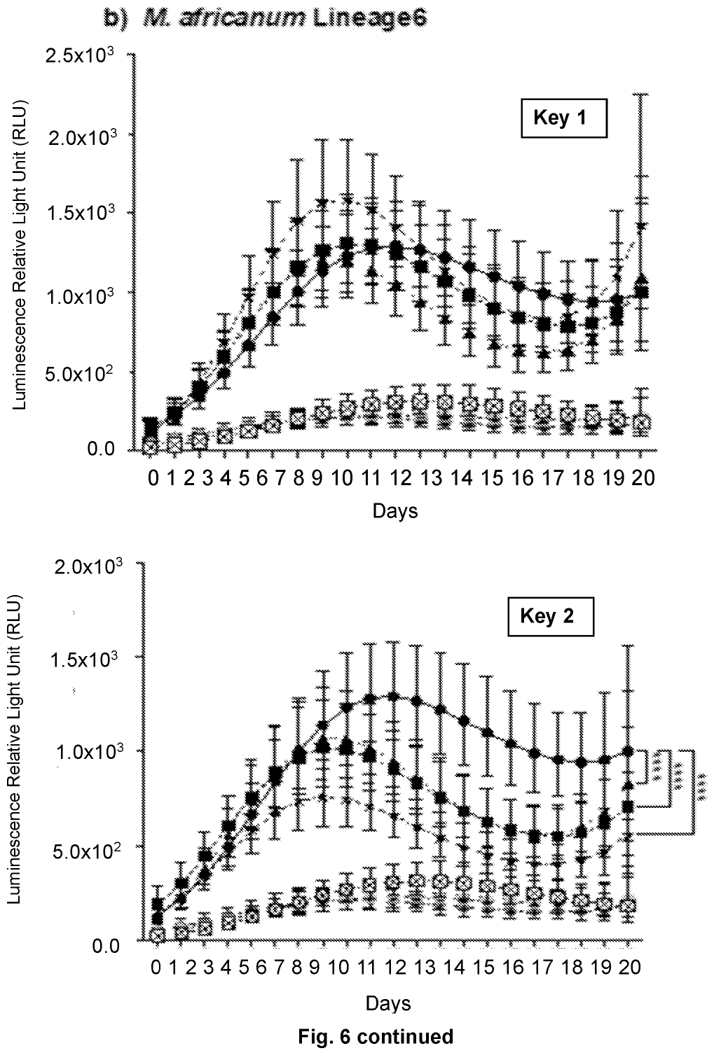

The selected MTBC lineages were cultured in the presence or absence of first-line anti-tuberculosis drugs (Isoniazid and Rifampicin; controls) and candidate host-directed therapy molecules on a 48 wells plate format (FIG. 1). The bacilli growth was monitored daily. The starting bacteria concentration of luminescence was 6000 relative light unit per millilitre (rlu/ml) concentration, which determines the required number of bacilli stock vials for each MTBC lineage. A luminescence of 6000 rlu/mL was equivalent to absorbance at optical density (OD 600 nm) to 0.011 and a McFarland of 0.11 on average of the different MTBC lineages tested.

Practically, the bacteria were thawed at room temperature; autoclaved beads were added to the vials (5-10 beads) and vortex thoroughly, then placed in a water bath sonicator for 15 minutes at room temperature to break the clumps. The declumped bacteria were diluted in the 7H9 culture media containing the selective antibiotics and Tween to the final concentration of 6000 rlu/ml. To each well of the 48 wells plate, 1 ml of diluted bacteria stock was added following a standardized plate plan. The following concentrations of 50 μM, 10 μM and 5 μM were tested for each HDT drug together with the control wells, including rifampicin (1 μg/ml) and isoniazid (0.4 μg/ml), and wells with just the bacteria without drugs, 7H9 medium without bacteria with and without HDT molecule. The plates were incubated at 37° C. and 70 rpm rocking incubator with daily bacteria growth measurements.

### Bacteria Growth Measurement

MTBC lineages growth in 48 wells culture plates were measured daily by reading the absorbance at optical density (OD 600 nm), luminescence (λ=578 nm) and GFP fluorescence (excitation and emission at 480 nm and 525 nm, respectively) on the SpectraMax® i3x Multi-Mode Microplate Reader (Molecular Devices). The first reading representing day zero (day 0) was done after 1-3 hours of drug assay set up; after that, the plates were read every 24 hours for the next 20 days. In parallel, the colony-forming units (CFU) were determined by subculture into 7H11 solid medium (Sigma Aldrich) supplemented with 10% oleic acid-albumin-dextrose-catalase (OADC, Becton Dickinson).

### Data Management and Visualization

The HDT drug test data is generated from the SpectraMax® i3x Multi-Mode Microplate Reader. The output from the machine is a .sda format which we convert to .xml format using basic visual programming. The .xml version is then managed using a Microsoft SQL Server(mssql) database organising the data into multiple tables, including all metadata from the machine. The database schema is designed to accommodate information related to the HDT experiment, including filename, assay plate design, drug concentrations, etc. The analysis then proceeds by extracting data via queries on the mssql database, merging different tables where necessary using the unique identifiers. The data analysis is performed using the R programming language. The ‘ggplot’ package in R is used to visualise the outcomes of interest, including absorbance, fluorescence, and luminescence over time (day). These were also plotted by experimental conditions, including M. tuberculosis complex lineages, HDT drug type, drug concentration and controls. Machine temperature is also extracted to help monitor the correct setup for the experiment at 37° C. All visualisation and daily monitoring/tracking of data from the experiment was packaged in a Shiny web application (https://shiny.rstudio.com/) hosted on an MRC server. This platform made data visualisation seamless for all lab staff facilitating access via a user-friendly interface to visually monitor the daily growth of bacteria under various experimental conditions. This platform consisted of the server-side, the data importation side, the user interface, and a global file where all the functions for data cleaning and filtration were contained. The tool addressed the challenge faced by lab staff automating the manual data extraction and the previous practice of ad hoc and inefficient visualisation approaches allowing monitoring of data in real-time.

### Statistical Analysis

We modelled each outcome variable (fluorescence, luminescence and CFU) using two-level random-intercept regression with a cubic function of time, accounting for within-replicates and within-plate variability occurring over time. The absorbance readout presented challenges to analyse directly due to the high number of out-of-range values, which requires an initial conversion into transmittance values. We used the logarithmic absorbance relationship to the Transmittance of the Beer-Lambert Law [Absorbance=−Log₁₀ Transmittance] to do the conversion. A logit transformation was then applied to the Transmittance and subsequently modelled using a two-level random-intercept regression with a cubic function of time, accounting for within-replicates and within-plate variability.

We log-transformed the outcome variables and generated a single variable containing all possible combinations of the different drugs, doses tested and bacteria. Interaction terms between this combined variable and time were included in the model.

Batches were set up in the laboratory on different days. Therefore, we adjusted the estimates for the experimental setup days to account for potential setup day effects. We calculated the predicted mean log-outcome (0 to 20 days) at each time point. We estimated the average drug's results for each MTBC lineage as a percentage change of the outcome (known as semi-elasticity) over 20 days. Of note, CFU data were collected every 48 hours from days 0 to 16. Due to multiple testing, we applied Dunn-Sidak's correction to control the familywise error rate (5 sub-analyses considered for the drug effect assessment per bacteria). The estimates, including their 95% confidence interval (95% CI), were plotted over time. The modelling and plots were done using Stata 17, the ‘ggplot’ package in R and GraphPad Prism version8.0, respectively.

### Example 3

### Calculation of MIC and IC₅₀ Data for Gefitinib, Erlotinib and Imatinib

### Materials and Methods

Following the methods as set out in the previous example, further drug concentrations of Imatinib, Erlotinib, and Gefitinib were tested against a prevalent MTBC lineage. 0.005 μM, 0.01 μM, 0.05 μM, 0.1 μM, 0.5 μM, 1 μM, 5 μM, 10 μM, 25 μM, 50 μM, 60 μM, 80 μM, 100 μM, 125 μM, 150 μM and 200 μM of each TKI were tested against Mtb-lineage2 to reliably determine the half maximal inhibitory concentration 50 (IC50) or half maximal effective concentration 50 (EC50) and minimal inhibitory concentration (MIC) of the TKI on each MTBC lineage.

### Results

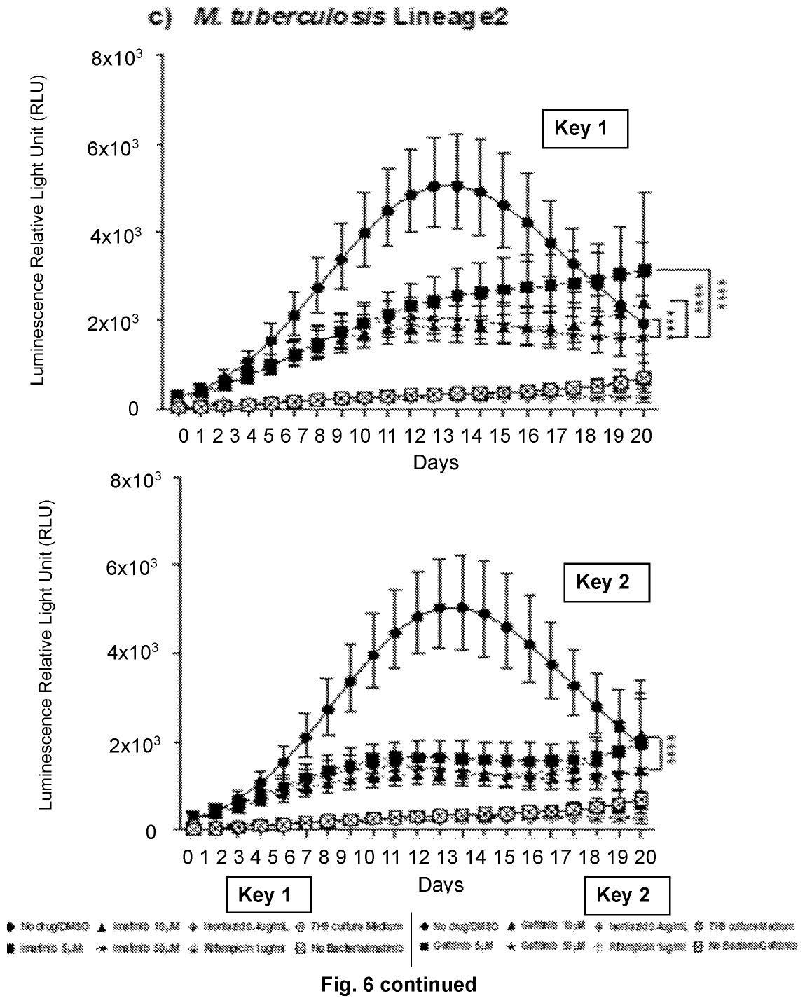

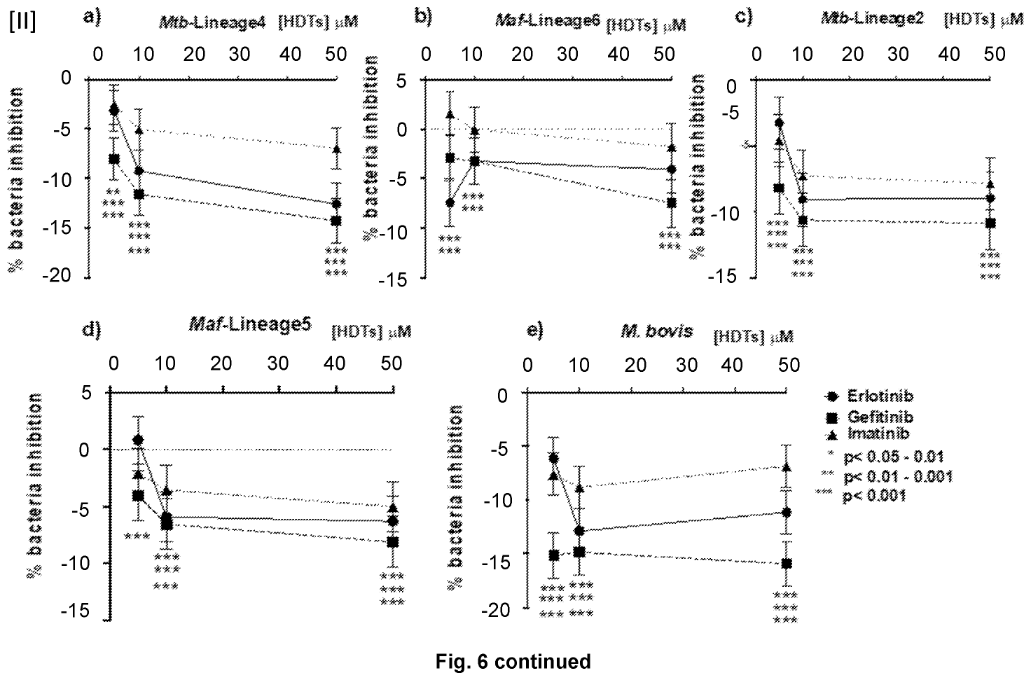

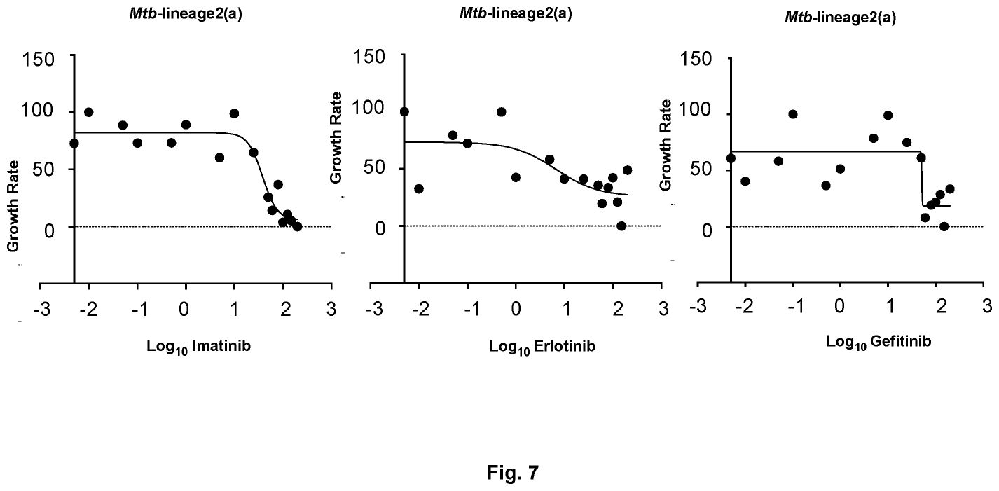

The MIC and IC50 were successfully calculated for each TKI against Mtb-lineage2 (FIG. 7, Table 1). There was significant variability between the MIC and IC50 values calculated among the bacterial lineages, highlighting that Maf-lineages and Mtb-lineage4 tended to have higher MIC and IC50 than other lineages. Erlotinib showed the lowest MIC and IC50 value when compared to gefitinib and imatinib.

TABLE 1
MIC and IC50 values for erlotinib, imatinib and gefitinib.

| Bacteria | | Bacteria | | Bacteria | |
|---|---|---|---|---|---|
| Drug | Erlotinib | Drug | Imatinib | Drug | Gefitinib |
| Parameter | MIC | Parameter | MIC | Parameter | MIC |
| Unit | (uM) | Unit | (uM) | Unit | (uM) |
| Day 15 | 32.97 | Day 10 | 56.05 | Day 10 | 54.28 |
| Day 16 | ~1.075 | Day 11 | 56.41 | Day 11 | 52.74 |
| Day 17 | 41.02 | Day 12 | 56.74 | Day 12 | 54.28 |
| Day 18 | 43.58 | Day 13 | 57.08 | Day 13 | 52.23 |
| Day 19 | 49.43 | Day 14 | 57.41 | Day 14 | 54.84 |

| Drug | | Drug | | Drug | |
|---|---|---|---|---|---|
| Parameter | IC50 | Parameter | IC50 | Parameter | IC50 |
| Day 15 | 16.27 | Day 10 | 32.8 | Day 10 | 50.03 |
| Day 16 | 10.06 | Day 11 | 33 | Day 11 | 50.13 |
| Day 17 | 8.653 | Day 12 | 33.21 | Day 12 | 50.22 |
| Day 18 | 7.614 | Day 13 | 33.45 | Day 13 | 50.22 |
| Day 19 | 6.79 | Day 14 | 33.74 | Day 14 | 50.56 |
| Day 20 | 6.186 | Day 15 | 34.12 | Day 15 | 50.24 |

### Example 4

### Direct Inhibitory Activity of Imatinib

### Materials and Methods

Following the methods as set in Example 2, nine clinical isolates were tested against imatinib, including the five previously reported in the discovery stage belonging to M. tuberculosis (Mtb)-lineage4, Mtb lineage2, M. africanum (Maf)-lineage5, Maf lineage6 and M. bovis. Adding isolates belonging to one of the above-listed lineages from different patients contributes to the diversity of the MTBC lineages tested, confirming that isolates belonging to different sublineages do not respond differently to the same drug.

The MIC and IC50 were determined from day 10 of the experiment.

### Results

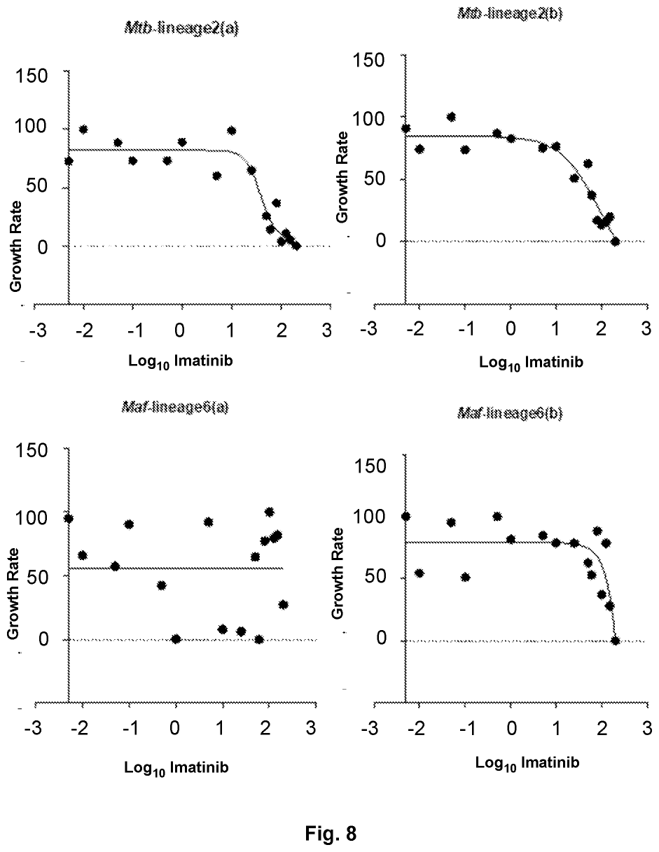

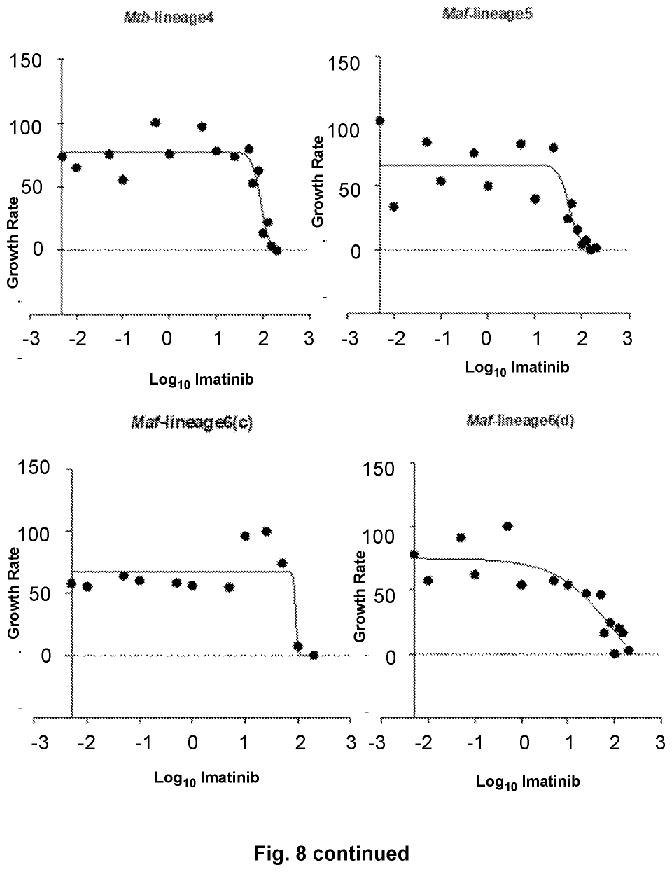

The results support the previous claims based on the fluorescence readout from Example 2. Mtb-lineage2, M. bovis, Maf-lineage5, Mtb-lineage4, and Maf-lineage6 (c) were found to be more susceptible to direct inhibition of Imatinib (FIG. 8, Table 2). However, three out of the four Maf-lineage6 isolates didn't respond to imatinib even at the highest concentration of 200 μM, and thus, the MIC and IC50 could not be calculated from them.

These results show the direct inhibitory activity for a further TKI, imatinib, against prevalent MTBC strains, highlighting their potential as a Mtb treatment against a wide range of lineages.

TABLE 2
MIC and IC50 values for imatinib showing direct inhibitory activity against prevalent MTBC strains.

| | Mtb- | Mtb- | | | | Maf- | Maf- | Maf- | Maf- |
|---|---|---|---|---|---|---|---|---|---|
| | lineage2 | lineage2 | Mtb- | | Maf- | lineage6 | lineage6 | lineage6 | lineage6 |
| Bacteria | (a) | (b) | lineage4 | M. bovis | lineage5 | (b) | (a) | (c) | (d) |
| Drug Parameter | MIC | MIC | MIC | MIC | MIC | MIC | MIC | MIC | MIC |
| Drug | IM | IM | IM | IM | IM | IM | IM | IM | IM |
| Unit | (uM) | (uM) | (uM) | (uM) | (uM) | (uM) | (uM) | (uM) | (uM) |
| Day 10 | 56.05 | 60.42 | 98.8 | 8.395 | 58.12 | ~9088 | ~0.7432 | 101 | ~46383 |
| Day 11 | 56.41 | 60.96 | Unstable | 16.26 | 59.49 | ~2393 | ~0.7212 | 100.6 | ~848356 |
| Day 12 | 56.74 | 60.68 | Unstable | 21.12 | 60.76 | 1464 | ~0.5938 | 100.3 | ~1470468 |
| Day 13 | 57.08 | 60.52 | Unstable | 25.55 | 62.01 | 1261 | ~0.5623 | 100.2 | ~5384506 |
| Day 14 | 57.41 | 85.42 | Unstable | 30.32 | 63.35 | 1521 | ~0.5412 | 100 | ~10083319 |
| Day 15 | 57.76 | 86.68 | 8.666E−246 | 35.21 | 65.2 | ~3586 | ~0.5149 | 100.1 | ~26363883 |

| Drug Parameter | IC50 | IC50 | IC50 | IC50 | IC50 | IC50 | IC50 | IC50 | IC50 |
|---|---|---|---|---|---|---|---|---|---|
| Day 10 | 32.8 | 71.52 | 50.44 | 1.904 | 50.31 | 1085 | 0.5195 | 88.04 | 13835 |
| Day 11 | 33 | 57.08 | 7547105 | 1.748 | 50.02 | 822.3 | Unstable | 88.75 | 658548 |
| Day 12 | 33.21 | 59.19 | 148.6 | 1.776 | 49.48 | 761 | Unstable | 88.82 | 1695736 |
| Day 13 | 33.45 | 59.43 | 148.3 | 1.903 | 48.62 | 821.1 | Unstable | 88.91 | 3092895 |
| Day 14 | 33.74 | 59.75 | 148.7 | 2.113 | 48.11 | 1073 | 0.483 | 88.99 | 6230506 |
| Day 15 | 34.12 | 59.98 | Unstable | 2.402 | 48.08 | 2158 | 2.865E−09 | 90.65 | 15270204 |

1-49. (canceled)

50. A method of treating tuberculosis, the method comprising providing to a subject requiring such treatment an amount of a protein tyrosine kinase inhibitor that is:
sufficient to directly inhibit growth of a Mycobacterium tuberculosis complex species, and
is lower than a dose of the protein tyrosine kinase inhibitor therapeutically effective as a host-directed therapeutic agent.

51. A method of treating tuberculosis according to claim 50, wherein the therapeutically effective amount of the protein tyrosine kinase inhibitor is a dose sufficient to establish in a recipient a serum concentration of the inhibitor that is between about 20 μM and about 100 μM.

52. A method of treating tuberculosis according to claim 50, wherein the therapeutically effective amount of the protein tyrosine kinase inhibitor is a dose sufficient to establish in a recipient a serum concentration of the inhibitor that is between about 30 μM and about 90 μM.

53. A method of treating tuberculosis according to claim 50, wherein the therapeutically effective amount of the protein tyrosine kinase inhibitor is a dose sufficient to establish in a recipient a serum concentration of the inhibitor that is between about 40 μM and about 80 μM.

54. A method of treating tuberculosis according to claim 50, wherein the therapeutically effective amount of the protein tyrosine kinase inhibitor is a dose sufficient to establish in a recipient a serum concentration of the inhibitor that is between about 50 μM and about 70 μM.

55. A method of treating tuberculosis according to claim 50, wherein the therapeutically effective amount of the protein tyrosine kinase inhibitor is a dose sufficient to establish in a recipient a serum concentration of the inhibitor of 150 μM or less, 100 μM or less, 90 μM or less, or 60 μM or less.

56. (canceled)

57. (canceled)

58. (canceled)

59. A method of treating tuberculosis, the method comprising providing to a subject requiring such treatment an amount of gefitinib that is sufficient to directly inhibit the growth of a Mycobacterium tuberculosis complex species.

60. A method of treating tuberculosis according to claim 59, wherein the therapeutically effective amount of gefitinib is a dose sufficient to establish in a recipient a serum concentration that is between about 50 μM and about 55 μM.

61. (canceled)

62. (canceled)

63. A method of treating tuberculosis, the method comprising providing to a subject requiring such treatment an amount of imatinib that is sufficient to directly inhibit the growth of a Mycobacterium tuberculosis complex species.

64. A method of treating tuberculosis according to claim 57, wherein the therapeutically effective amount of imatinib is a dose sufficient to establish in a recipient a serum concentration that is between about 20 μM and about 100 μM.

65-75. (canceled)

76. A method of treating tuberculosis according to claim 59, wherein the gefitinib is used as a broad-spectrum tuberculosis treatment.

77. A method of treating tuberculosis according to claim 59, wherein the Mycobacterium tuberculosis complex species growth of which is inhibited is selected from the group consisting of: Mycobacterium tuberculosis (such as M. tuberculosis-lineage2), Mycobacterium africanum, Mycobacterium bovis and combinations thereof.

78. A method of treating tuberculosis according to claim 59, wherein the tuberculosis treated is multi-drug resistant (MDR) tuberculosis.

79. A method of treating tuberculosis according to claim 59, wherein the tuberculosis has been identified as being caused by infection with Mycobacterium tuberculosis-lineage2.

80. A method of treating tuberculosis according to claim 59, wherein the tuberculosis has been identified as being caused by infection with Mycobacterium africanum or Mycobacterium bovis.

81. A method of treating tuberculosis according to claim 63, wherein the Mycobacterium tuberculosis complex species growth of which is inhibited is selected from the group consisting of Mycobacterium tuberculosis-lineage2, Mycobacterium tuberculosis-lineage4, Mycobacterium africanum-lineage5, Mycobacterium africanum-lineage6, Mycobacterium bovis and combinations thereof.

82. A method of treating tuberculosis according to claim 63, wherein the tuberculosis has been identified as caused by infection with a Mycobacterium tuberculosis complex species selected from the group consisting of Mycobacterium tuberculosis-lineage2, Mycobacterium tuberculosis-lineage4, Mycobacterium africanum-lineage5, Mycobacterium africanum-lineage6, Mycobacterium bovis and combinations thereof.
